(19) United States
(12) Patent Application Publication
(10) Pub. No.: US 2013/0085462 A1
(43) Pub. Date: Apr. 4, 2013

(54) ELECTROKINETIC PUMP BASED WOUND TREATMENT SYSTEM AND METHODS

(71) Applicants: Kenneth Kei-ho NIP, Redwood City, CA (US); Jessica L. Strohmann, Fremont, CA (US); Doris Sun-Chia Shieh, Santa Clara, CA (US); Tuan Quoc Mai, San Ramon, CA (US); Robert B. Lewis, Pleasanton, CA (US); Kenneth R. Hencken, Pleasanton, CA (US); Craig S. Bryant, Alameda, CA (US)

(72) Inventors: Kenneth Kei-ho NIP, Redwood City, CA (US); Jessica L. Strohmann, Fremont, CA (US); Doris Sun-Chia Shieh, Santa Clara, CA (US); Tuan Quoc Mai, San Ramon, CA (US); Robert B. Lewis, Pleasanton, CA (US); Kenneth R. Hencken, Pleasanton, CA (US); Craig S. Bryant, Alameda, CA (US)

(21) Appl. No.: 13/632,884

(22) Filed: Oct. 1, 2012

Related U.S. Application Data
(60) Provisional application No. 61/541,988, filed on Sep. 30, 2011.
(60) Provisional application No. 61/576,930, filed on Dec. 16, 2011.

(51) Int. Cl. A61M 35/00; A61M 1/00

(52) U.S. Cl. 604/315

(57) ABSTRACT

A wound treatment system includes a patch, first and second fluid reservoirs, an electrokinetic pump assembly, and a controller. The patch is configured to enclose a wound area and includes an inlet and an outlet. The first fluid reservoir is fluidically connected to the inlet and the second fluid reservoir is fluidically connected to the outlet. The electrokinetic pump assembly is configured to pump a first treatment fluid from the first fluid reservoir into the patch through the inlet and to pump fluid from the patch through the outlet and into the second fluid reservoir. The controller is configured to operate the electrokinetic pump assembly and to include an electronic memory containing computer readable instructions for operating the electrokinetic pump assembly to perform a wound therapy protocol in the wound area.

### CROSS REFERENCE TO RELATED APPLICATIONS

This application claims priority to U.S. Provisional Application No. 61/541,988, filed Sep. 30, 2011, and titled “ELECTROKINETIC PUMP BASED WOUND TREATMENT SYSTEM AND METHODS,” and to U.S. Provisional Application No. 61/576,930, filed Dec. 16, 2011, and titled “ELECTROKINETIC PUMP BASED WOUND TREATMENT SYSTEM AND METHODS,” both of which are herein incorporated by reference in their entireties.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application relates generally to systems and methods of closed wound treatment systems to promote wound healing. In particular, this disclosure describes liquid and pressure tight patches and pumps suited for managing both irrigation of a wound treatment area as well as removal and/or evacuation of the wound site. In particular, the pumps used to provide wound treatment therapy are electrokinetic pumps.

### BACKGROUND

Wounds occur when the integrity of tissue is compromised, affecting one or more layers of the epidermis or underlying tissue. Acute wounds may be caused by an initiating event, such as an accident-related injury or surgical procedure or by operation of an infectious disease, and generally take the form of punctures, abrasions, cuts, lacerations, or burns. Chronic wounds are wounds that generally do not heal within three months due to one or more of: ischemia of the vessels supplying the tissue, venous hypertension or compromise of the immune response, such as observed, for example, with venous ulcers, diabetic ulcers and pressure ulcers. Depending on etiology, such as diabetes, venous insufficiency, or cardiovascular failures, acute wounds may become recalcitrant and even chronic.

The introduction of bacteria from external sources into the wound typically causes inflammation that activates the patient's immune response, in turn causing white blood cells, including neutrophil granulocytes, to migrate towards the source of inflammation. While they fight pathogens, such neutrophils also release inflammatory cytokines and enzymes that damage cells. In particular, the neutrophils produce an enzyme called myeloperoxidase that in turn is metabolized to produce reactive oxygen species that kill bacteria. Collaterally, such enzymes and reactive oxygen species damage cells in the margin surrounding the wound, referred to as the “periwound,” thereby preventing cell proliferation and wound closure by damaging DNA, lipids, proteins, the extracellular matrix and cytokines that facilitate healing. Because neutrophils remain in chronic wounds for longer than in acute wounds, they contribute to higher levels of inflammation. Moreover, the persisting inflammatory phase in chronic wounds contributes to exudate (fluid that flows from the wound) with high concentrations of matrix metalloproteases (MMPs). Excess MMPs results in degradation of extracellular matrix protein. In addition to damaging the wound, exudate damages the periwound tissue exposed to it as well. In particular, exudate that flows out of the wound and onto periwound region may damage the fragile skin, which is already compromised due to the patient's underlying etiology, such as diabetes. Such damage may degrade the periwound skin and cause its breakdown and turn it into a wound. Thus, exudate flow onto the periwound region will cause many complications, including the potential for increasing the size of the wound and prolonging its healing. Such damage to the skin in the periwound region (periwound skin) makes it more susceptible to tearing and resultant intense pain as dressings or devices adhered to them are removed. Other complications include infection of the periwound region and intense itching.

Patients suffering from chronic wounds frequently report experiencing severe and persistent pain associated with such wounds, which may arise from necrosis of and/or nerve damage of the skin and underlying tissue. Treatment for such pain often consists of low dose analgesics, while topical antibiotics and/or debridement, which seeks to remove necrotic tissue from the wound, may be used to control the bacterial load at the wound site.

Conventional wound treatment also typically involves covering the wound with a dressing to prevent further contamination and infection, to retain moisture, and to absorb exudate. While exudate contains biochemical compounds that benefit wound healing as noted above, its excessive amount in wound or its presence in the periwound region facilitates degradation of tissue, and the exudate additionally serves as a growth medium for bacteria. The consistency of exudate varies, depending on the type of wound and the stage of healing. For example, exudate may be watery, extremely viscous, or somewhere in between. Moreover, the sizes of wounds can vary greatly, as can their care.

Although a wide variety of dressings have been developed, few previously-known wound treatment systems properly manage exudate, e.g., removing a sufficient amount of exudate from the wound site and/or while protecting the periwound region from damaging contact with the exudate. Moreover, conventional systems typically do not address the pain created by the wound treatment system, particularly where the wound treatment system continuously contacts the wound. For example, gauze, which is applied directly onto a wound, is capable of absorbing only a limited amount of exudate, and readily transports excess exudate onto the periwound region, causing maceration and damage. Moreover, the gauze typically is in direct contact with the wound and adheres to it, so that normal motion of the patient results in rubbing, itching and discomfort. In addition, removal of the gauze at periodic intervals is painful and frequently disrupts any healing that may have occurred.

Some previously-known approaches to wound treatment attempt to reduce adhesion between the wound and the dressing by applying additional substances. For example, the wound and dressing may be soaked in saline water to loosen adherence and/or soften any scabs that formed, thus facilitating removal of the dressing. Or, for example, antibiotic ointments such as polymyxin B sulfate or bacitracin can be applied to reduce sticking. However, such methods are not always satisfactory because soaking a particular wound in water or applying ointments may not be practical or recommended.

Some previously-known dressings are promoted as being “non-stick” or “non-adherent” may be composed of materials such as hydrocolloids, alginates, and hydrofilms. Regardless of the low level of adherence of such dressings to the wound, continuous contact between the dressing and wound disturbs the fragile wound matrix, and may undermine the growth of blood vessels and epithelial cells in the wound bed. Such disturbance often occurs when the dressing is removed, or simply as a result of the contact between the bandaged area and the patient's environment. Pain is often concomitant with such disturbances. In addition, previously-known “non-stick” dressings usually do not absorb sufficient amounts of exudate, and thus require frequent monitoring and changing. These drawbacks add to the cost of use and limit the applicability of such previously-known wound treatment systems.

Some previously-known dressings are design to manage exudate but provide either limited benefit and/or at a much higher perceived cost. For example, a foam dressing is designed to absorb large amounts of exudate. However, use of this product is restricted to highly exuding wounds because its highly absorptive properties can result in desiccation of wounds that are not highly exuding, thereby impeding healing. In addition, because foam dressings cannot be conformed to the size and shape of the wound, the dressing typically overlaps with the periwound region. Consequently, exudate absorbed by the foam is transported throughout the foam and onto the periwound region, where prolonged exposure leads to maceration and degradation of the periwound region. Other previously-known dressings, such as a hydrofiber dressing contact the wound bed, and are intended to absorb exudate and transfer and sequester the exudate in a layer disposed atop the wound. This and similar previously-known dressings do not entirely contain or absorb exudate. Moreover, like foam and other previously-known dressings, a hydrofiber dressing essentially plugs the wound surface and creates an osmotic environment in which the fluidic osmotic pressure within the wound bed approximates that of the surrounding tissue. Consequently, exudate is not sufficiently drawn from the wound, and its buildup in the wound may adversely affect the wound and periwound region. Furthermore, previously-known dressings do not provide an adequate moisture vapor transfer rate (MVTR) away from the wound environment, thus creating the potential for an over-hydrated environment that hinders wound healing.

Other previously-known wound treatment systems, employ a mechanically operated contact-based dressing that continuously vacuums exudate from the wound bed. It and other dressings incorporating the concept of Negative Pressure Wound Therapy (NPWT) have proven particularly useful in healing large wounds, such as surgical wounds. However, such systems are costly, difficult to apply, and time consuming. In addition, some such systems require insertion of a sponge or gauze directly into the wound bed, they cause considerable pain and discomfort for the patient and are not be appropriate for many types of wounds.

In addition, several previously-known dressings have been developed that are promoted as “non-contact” dressings, which seek to prevent adhesion of the wound tissue to dressing, or to facilitate treatment without contacting the wound. Dressings such as these are commonly formed as an inverted cup or a raised bandage with limited deformability to cover the wound without contacting it. Conventional pumping and/or vacuum systems—along with their requisite power and control system requirements—have been suggested for use with these conventional non-contact dressings. However, such previously-known dressings and systems have not adequately addressed the needs of promoting wound healing while also facilitating protection of the periwound region.

What is needed are simplified pumping systems operating with improved non-contact wound patches to provide a wound treatment system with enhanced capabilities to provide positive and negative pressure based wound therapy.

### SUMMARY OF THE DISCLOSURE

In general, in one embodiment, a wound treatment system includes a patch, first and second fluid reservoirs, an electrokinetic pump assembly, and a controller. The patch is configured to enclose a wound area and includes an inlet and an outlet. The first fluid reservoir is fluidically connected to the inlet and the second fluid reservoir is fluidically connected to the outlet. The electrokinetic pump assembly is configured to pump a first treatment fluid from the first fluid reservoir into the patch through the inlet and to pump fluid from the patch through the outlet and into the second fluid reservoir. The controller is configured to operate the electrokinetic pump assembly and to include an electronic memory containing computer readable instructions for operating the electrokinetic pump assembly to perform a wound therapy protocol in the wound area.

This and other embodiments can include one or more of the following features.

The wound therapy protocol can provide for an amount of the contents of the first reservoir to be delivered to the wound area. The wound therapy protocol can provide for a time duration that a portion of the contents of the first reservoir are to remain in the wound area. The wound therapy protocol can provide for a time duration for operation of the electrokinetic pump assembly to pump substantially all of the contents of the wound area to the second reservoir. The wound therapy protocol can provide for a time duration for the operation of the electrokinetic pump assembly depending upon the contents of the first reservoir. The wound therapy protocol can provide for a time duration for the operation of the electrokinetic pump assembly depending upon the fluid contents of the wound area. The fluid contents of the wound area can be related to the contents of the first reservoir in the wound area. The fluid contents of the wound area can be related to a volume of fluid in the wound area.

The controller can be configured to estimate a volume of fluid taken from the first reservoir, a volume of fluid removed from the patch area, or a volume of fluid pumped into the second reservoir. The controller can be configured to estimate a volume based upon a number of cycles of the electrokinetic pump assembly operation.

The electrokinetic pump assembly can weigh less than 75 grams. The wound treatment system including the reservoirs, the pump assembly, the controller, and a power source have a volume of less than 100 cubic inches. The wound treatment system can be configured to be attached and carried on a patient.

The wound treatment system can further include a container, the container including both the first and second reservoirs. The container can have a movable member therein to separate the first fluid reservoir from the second reservoir. The movable member can be configured such that the volume of the first reservoir decreases while the volume of the second reservoir increases. The wound treatment system can further include a second container having a third reservoir and a fourth reservoir, and wherein the second container is configured to be interchangeable with the first container such that a second treatment fluid can be pumped by the electrokinetic pump into the patch from the third reservoir and fluid can be pumped from the patch into the fourth fluid reservoir.

The electrokinetic pump assembly can include two electrokinetic pumps, one electrokinetic pump configured to pump fluid from the first fluid reservoir into the patch and the second electrokinetic pump configured to pump fluid from the patch into the second fluid reservoir. The computer readable instructions can provide for the two pumps to run at substantially the same time. The computer readable instructions can provide for the two pumps to run on separate pumping cycles. The computer readable instructions can provide for one of the two pumps to operate depending upon the contents of the first reservoir. The computer readable instructions can provide for one of the two pumps to operate depending upon a duration that a portion of the contents of the first reservoir has remained within the wound area. The computer readable instructions can provide for one of the two pumps to operate depending upon a volume of fluid contained within the wound area.

The wound treatment system can further include a sensor configured to measure the pressure inside the patch. The wound treatment system can further include a controller configured to pump fluid in or out based upon the pressure.

The computer readable instructions can further include an instruction to operate the electrokinetic pump assembly such that fluid is moved in and out of the wound area at predetermined time intervals.

The system can be configured to operate the electrokinetic pump assembly maintain the pressure under the patch at under 0.8 psi. The system can be configured to operate the electrokinetic pump assembly to maintain the pressure under the patch at greater than or equal to −5 psi.

The computer readable instructions can further include an instruction to operate the electrokinetic pump assembly to maintain a volume of fluid in the wound area below a total volume of an enclosed wound area. The computer readable instructions can further include an instruction to operate the electrokinetic pump assembly to maintain a volume of fluid in the wound area as defined in a wound treatment protocol.

The patch can include a movable film and a protective shell. The wound treatment system can further include a bypass check valve in communication with the wound area and the second fluid reservoir with a setting to open when the pressure within the wound area reaches a set point selected to prevent loss of a sealing along the enclosed wound area.

The wound treatment system can be configured to deliver a minimum dose of the contents of the first reservoir of less than 1 ml. The minimum dose can have a volume of less than 0.5 ml. The minimum dose can have a volume of less than 0.1 ml. The system can be configured to deliver a dose of the contents of the first reservoir with an incremental dose adjustment of less 0.5 ml. The incremental dose adjustment can be less than 0.1 ml.

The system can further include a battery configured to run the electrokinetic pump assembly. The battery can be configured to run the electrokinetic pump assembly for over 48 hours without charging. The battery, patch, and pump assembly weigh less than 450 grams. The battery can be a rechargeable battery.

The system can further include an AC adapter for powering the electrokinetic pump assembly.

The system can further include at least one quick disconnect mechanism configured to disconnect the patch from the first and second fluid reservoirs such that third and fourth fluid reservoirs can be attached to the patch. The quick disconnect can be between the patch and the electrokinetic pump assembly. The quick disconnect can be between the electrokinetic pump assembly and the first and second reservoirs.

In general, in one embodiment, a method of providing a wet wound therapy to a sealed wound treatment volume includes: operating an electrokinetic pumping system to supply a treatment fluid into the sealed wound treatment volume; operating an electrokinetic pumping system to remove a fluid from the sealed wound treatment volume; and performing the step to supply and the step to remove during a period of at least 24 hours without removing a patch used to form a perimeter of the sealed wound treatment volume.

This and other embodiments can include one or more of the following features. The performing step can be performed during a period of at least 48 hours without removing the patch. The performing step can be performed during a period of at least 72 hours without removing the patch. The performing step to supply can include delivering the same treatment fluid. The performing step to supply can include delivering a second, different treatment fluid. The method can further include: before performing the step to supply a second treatment fluid, disconnecting a first reservoir containing a first treatment fluid from the electrokinetic pump to supply; and connecting a second reservoir containing the second treatment fluid to the electrokinetic pump to supply. The first treatment fluid can include saline, an antimicrobial mixture, or a growth promoting drug.

In general, in one aspect, a method of providing a wet wound therapy to a patient includes: attaching a wound care patch to a patient to form a sealed perimeter and a wound treatment volume about a wound on the patient; establishing a fluid circuit between an electrokinetic pump assembly, at least one fluid reservoir, and the wound treatment volume; attaching at least the electrokinetic pump assembly to the patient; and operating the electrokinetic pump assembly to move a fluid through the fluid circuit between the at least one reservoir and the wound treatment volume.

This and other embodiments can include one or more of the following features. Operation of the electrokinetic pump assembly can move fluid from the at least one reservoir into the wound treatment volume. Operation of the electrokinetic pump assembly can move fluid from the wound treatment volume into the at least one reservoir. The rate of moving fluid through the fluid circuit can be metered in increments of less than 1 ml, such as less than 0.5 ml or less than 0.1 ml. The at least one fluid reservoir can be a first reservoir and a second reservoir, and the step of operating the electrokinetic pump assembly can move fluid through the fluid circuit from the first reservoir to the wound treatment volume and through the fluid circuit from the wound treatment volume to the second fluid reservoir. The step of moving fluid to the wound treatment volume can occur at a different time than the step of move fluid from the wound treatment volume. The step of moving fluid from the wound treatment volume can be performed to remove 40-80% of a fluid present in the wound treatment volume. The method can further include: operating the electrokinetic pump to move fluid through the fluid circuit from the first reservoir to the wound treatment volume after the removing a fluid present in the wound treatment volume. The electrokinetic pump assembly can further include a first electrokinetic pump configured to move fluid through the fluid circuit from the first reservoir to the wound treatment volume and a second electrokinetic pump configured to move fluid through the fluid circuit from the wound treatment volume to the second fluid reservoir. The duration of operating of the first and the second electrokinetic pumps can be selected from a pre-determined wound treatment protocol. The method can further include measuring a pressure related to the wound treatment volume and performing the operating the electrokinetic pump based on the measured pressure. The performing step can include moving fluid into the wound treatment volume. The performing step can include moving fluid from the wound treatment volume. The method can further include: performing the operating the electrokinetic pump to maintain the measured pressure in relation to a setpoint determined by a wound therapy protocol. The setpoint can be less than 0.8 psi. The setpoint can be greater than −5 psi.

In general, in one embodiment, a method of wet wound therapy includes: delivering a dose of a treatment fluid to a wound area that is sealed with a patch; and activating a negative pressure of at least −1 psi under the patch, wherein the delivering and activating are performed without removing the patch.

This and other embodiments can include one or more of the following features. The method can further include removing waste from the treatment site. The method can further include maintaining a negative pressure of between −1 psi and −5 psi during a portion of a wound therapy. The delivering and activating steps can be performed with an electrokinetic pump assembly. The method can further include activating the negative pressure without touching a top of the patch to the wound area.

In general, in one aspect, a wound treatment system includes a patch, first and second fluid reservoirs, and an electrokinetic pump assembly. The patch is configured to enclose a wound area and have an inlet and an outlet. The first fluid reservoir is fluidically connected to the inlet and the second fluid reservoir is fluidically connected to the outlet. The electrokinetic pump assembly is configured to pump a first treatment fluid from the first fluid reservoir into the patch through the inlet and to pump fluid from the patch through the outlet and into the second fluid reservoir, the pump assembly further configured to maintain a negative pressure under the patch. The pump assembly can be configured to maintain the a negative pressure of between −1 psi to −5 psi.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a schematic view of a closed wound treatment system having a patch and two separately controlled electrokinetic pumps;

FIG. 1B is a schematic view of a closed wound treatment system having a patch and two electrokinetic pumps in a master slave configuration;

FIG. 1C is a schematic view of a closed wound treatment system having a patch and two electrokinetic pumps controlled by a single controller;

FIG. 2A is a schematic view of a closed wound treatment system having a patch and a single reciprocating electrokinetic pump system;

FIG. 2B is a schematic view of a closed wound treatment system having a patch and a single direct drive electrokinetic pump system;

FIG. 3 is a flowchart of a method of providing fluid to and from a wound patch treatment area;

FIG. 4 is a schematic view of a closed wound treatment area;

FIG. 4A illustrates the result of the evacuation cycle operation on a patch and wound treatment volume;

FIG. 4B illustrates the result of the delivery cycle operation on a patch and wound treatment volume;

FIGS. 5A, 5B and 5C are top, section, and bottom views, respectively, of an exemplary flat top wound patch;

FIGS. 6A, 6B and 6C are top, section, and bottom views, respectively, of an exemplary rounded top wound patch having internal reinforcement elements;

FIGS. 7A, 7B and 7C are top, section, and bottom views, respectively, of an exemplary rounded top wound patch having internal reinforcement elements;

FIGS. 8A, 8B and 8C are top, section, and bottom views, respectively, of an exemplary rounded top wound patch having external reinforcement elements;

FIGS. 9A, 9B and 9C are top, section, and bottom views, respectively, of an exemplary rounded top wound patch having external reinforcement elements;

FIG. 10 is a curve illustrating the percentage volume removed from the patch interior volume as a function of vacuum applied to the patch for the patch embodiments of FIGS. 5-9;

FIG. 11 is a curve illustrating the percentage volume removed from the patch interior volume as a function of vacuum applied to a patch having a durometer of 15 shoreA and worn by two different test subjects, to a patch having a durometer of 5 shoreA and worn by two different test subjects, and to a patch having a durometer of 5 shoreA having a mesh reinforcement member and worn by two different test subjects.

FIGS. 12A, 12B, and 12C show top, side, and sections views, respectively, of a rounded square patch;

FIGS. 13A, 13B, and 13C show top, section, and bottom views, respectively, of an exemplary patch having a reinforcement member therein;

FIGS. 14A and 14B show top and section views, respectively, of a patch having bumps on the upper inside surface;

FIG. 15 is a top down view of a patch having dissimilar inner and outer perimeters in place over a wound treatment site;

FIG. 16 is a schematic view of an electrokinetic pump powered wound treatment system that includes an exudate sampler;

FIG. 17A is a schematic view of a closed patch wound treatment system powered by an electrokinetic pump system and having a single divided container for both supply and collection fluids;

FIG. 17B is an enlarged view of the divided container of FIG. 17A;

FIG. 18 is an exemplary flowchart for providing therapy to a closed wound treatment site using a single electrokinetic pump;

FIGS. 19A-19D illustrate the system and valve configurations corresponding to the method described in FIG. 18;

FIG. 20 is a schematic view of a single pump system having a disinfectant supply and associated valves;

FIG. 21 shows a “negative pressure only” electrokinetic wound pump system;

FIGS. 22A and 22B show a patch having a strain gauge extending across the surface;

FIG. 23 illustrates a schematic view of a wound pump system having a pressure sensor and two check valves to determine the pressure under the patch;

FIG. 24 illustrates a schematic view of a wound pump system having a spring-loaded switch to determine the pressure under the patch;

FIG. 25 is a graph showing a system for turning the wound pump system on and off using pressure readings;

FIG. 26 illustrates an isometric view of a distribution manifold;

FIG. 27 illustrates an isometric view of a prototype of a two electrokinetic pump system;

FIG. 28 illustrates a schematic view of a prototype of a two electrokinetic pump—three patch treatment system;

FIG. 29 illustrates a schematic view of a wound treatment system configured to operate with a recirculation loop through a germicidal treatment component;

FIG. 30 illustrates a curve representing the germicidal effectiveness of various wavelengths of the ultraviolet radiation spectrum;

FIG. 31 is a section view of a patch having three LED type bulbs positioned to emit Ultraviolet (UV) radiation into the wound treatment volume; and

FIGS. 32A-32C illustrate various views of an exemplary portable wound treatment system including a waste reservoir, a treatment reservoir, two pumps, and an electronics package.

### DETAILED DESCRIPTION

Non-contact dressing or patch embodiments described herein have pre-formed shapes and sizes and are designed with enhanced deformability, thereby providing an ability to control exposure of the periwound skin to exudate. Additionally, the enhanced deformability and variable adhesion layer capabilities of the patch embodiments described herein enable application of the patches to small surface wounds or wound areas with complex topology, such as the ankle or foot. In addition, non-contact wound treatment systems described herein manage and control the periwound region environment including providing a wide range of positive and negative pressures. As a result, the formation of pressure rings around the wound may be reduced, thereby reduced ischemia in the wound and surrounding tissue. Importantly, the patch and system controls described herein provide a variety of mechanisms to stimulate the flow of exudate and/or sequester exudate away from the wound in therapeutically relevant volumes. Moreover, the fluid and pressure control aspects of the inventive methods and systems may also be used to manage humidity about the wound and periwound region, thereby reducing the onset of maceration and/or periwound degradation while enhancing the healing process.

### System Design

The closed wound treatment systems described herein can include a patch and a pump assembly connected through a fluid circuit to deliver and evacuate fluid from the patch. The pump assembly can include single pumping system to both evacuate and deliver the fluid or separate evacuation and delivery pump systems.

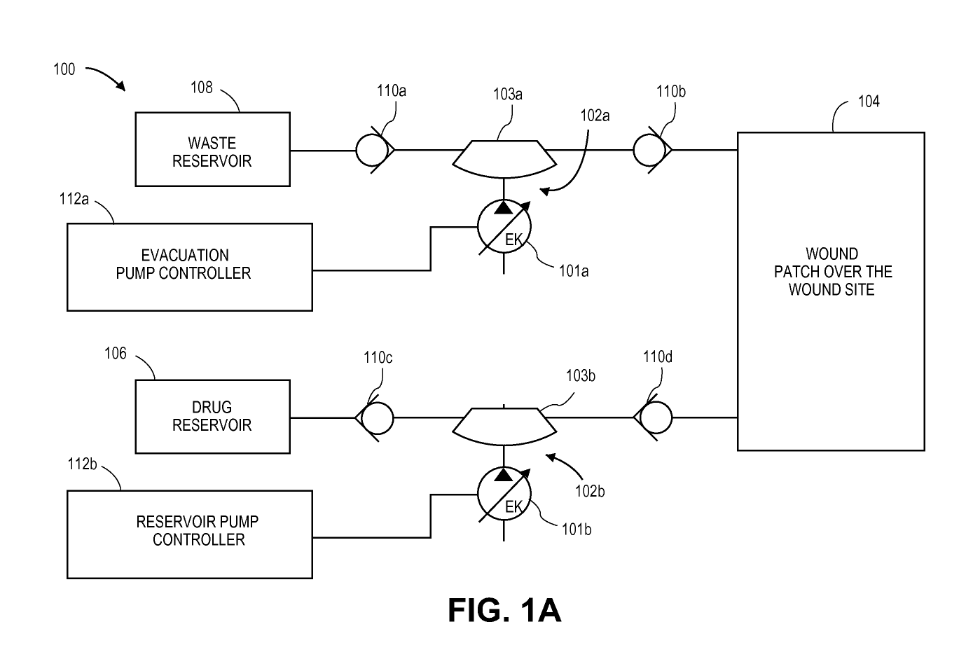

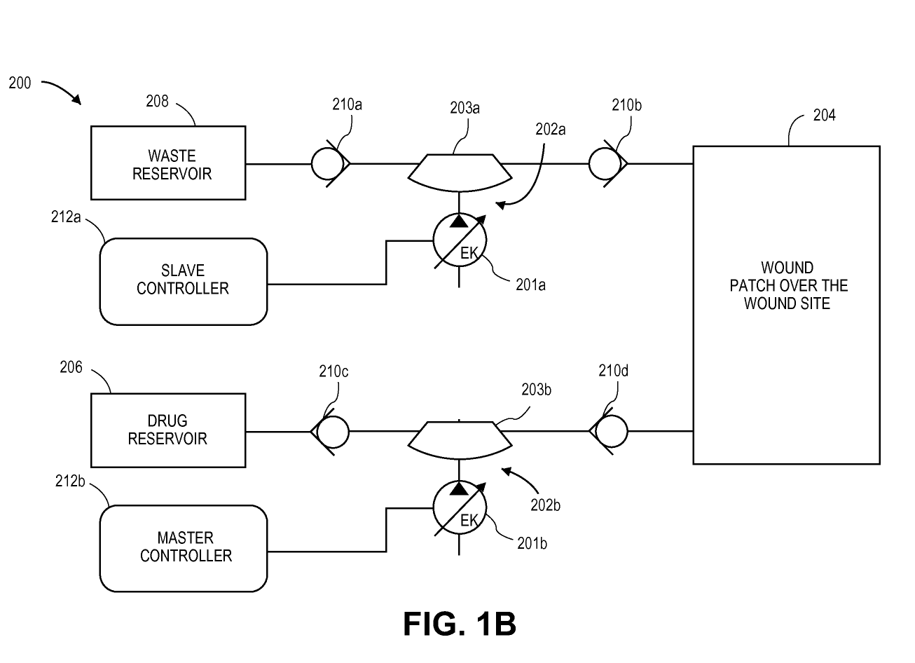

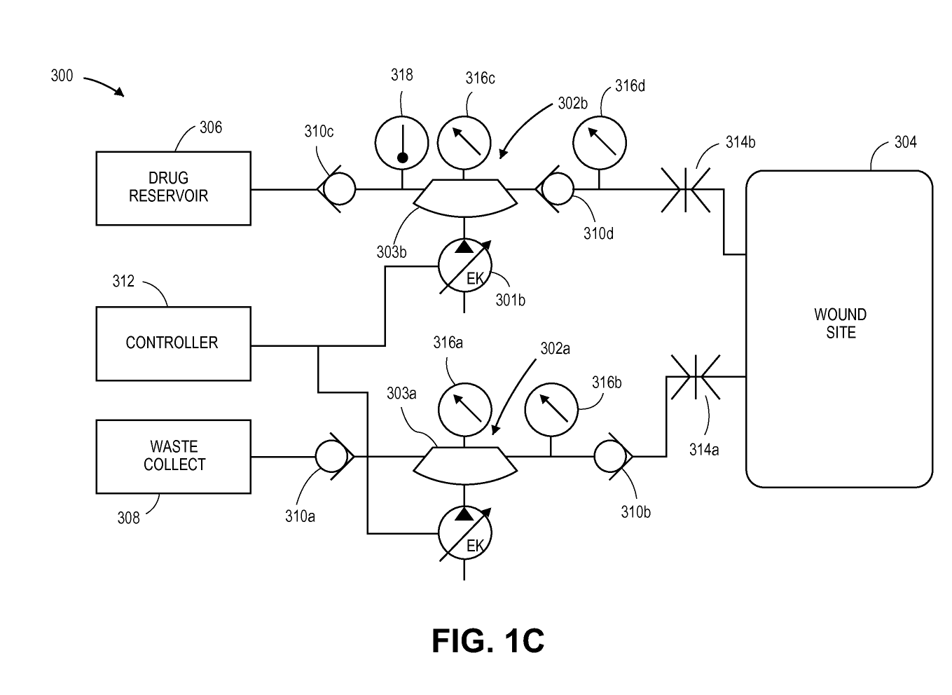

Referring to FIGS. 1A-1C, closed wound treatment systems can include a patch and two separate pumping systems—one for delivering fluid to the wound site and one for evacuating the patch.

FIG. 1A is a schematic view of a closed wound treatment system 100 having a patch 104 and two separately controlled electrokinetic pump systems 102a, 102b (each having a respective electrokinetic engine 101a,b and pump 103a,b). The pump system 102b can supply fluid from a reservoir 106, such as a drug reservoir, to the wound treatment site under the patch 104. The pump system 102a can evacuate fluid from the wound treatment site and pass it into a reservoir 108, such as a waste reservoir. The system 100 can further include inlet and outlet check valves 110a,b,c,d to control the amount of fluid under the patch 104. In some embodiments, additional bypass check valves can be present to provide a back-up if one of the pumps stops working, e.g., the bypass valve can open if or when pressure within the wound area reaches a set point selected to prevent loss of a sealing along the enclosed wound area. As shown in FIG. 1A, each pump system 102a,b can have a separate controller 112a,b to control the amount of fluid pumped in and out of the wound site under the patch 104.

FIG. 1B shows a closed wound treatment system 200 having pump controllers 212a,b in a master slave configuration. Like the wound treatment system 100, wound treatment system 200 includes a patch 204, two electrokinetic pump systems 202a,b (each having a respective electrokinetic engine 201a,b and pump 203a,b), a drug reservoir 206, a waste reservoir 208, and inlet and outlet check valves 210a,b,c,d.

FIG. 1C shows a wound treatment system 300 having a single controller 312 to control two pumps 302a,b. The single controller 312 can include, for example, two H-bridges that allow for control of both the delivery and the evacuation pump systems 302a,b. Like the wound treatment systems 100,200, wound treatment system 300 includes a patch 304, two electrokinetic pump systems 302a,b (with respective electrokinetic engines 301a,b and pumps 303a,b), a drug reservoir 306, a waste reservoir 308, and inlet and outlet check valves 210a,b,c,d.

As shown in FIG. 1C, the wound treatment system 300 can further include one or more quick disconnects 314a,b, such as a luer lock. The quick disconnects 314a,b, can be configured to allow the reservoirs 306, 308 to be easily disconnected from the patch 304, thereby allowing for use of new reservoirs including, for example, reservoirs containing different treatment fluids. The quick disconnects 314a,b can be located directly between the patch 314 and the pump systems 302a,b and/or directly between the pump systems 302a,b and the reservoirs 306, 308. One or more quick disconnects 314a,b can similarly be used in wound treatment systems 100, 200.

As shown in FIG. 1C, the system 300 can further include pressure sensors 316a,b associated with pump system 302a and pressure sensors 316c,d associated with pump system 302b. As discussed further below, the pressure sensors 316a,b,c,d can be used to regulate the amount of fluid either delivered from the drug reservoir 306 or evacuated from the patch 304. Pressure sensors 316a,b,c,d can likewise be used for wound treatment system 100, 200.

Referring still to FIG. 1C, the system 300 can further include a temperature sensor 318 configured to measure the temperature, for example to determine whether a chance in viscosity has occurred as a result of temperature. The results of the temperature sensor can then be used to adjust the wound care protocol accordingly. Temperature sensors, pressure sensors, and other feedback loops are described in copending U.S. patent application Ser. No. 13/465,902, titled “SYSTEM AND METHOD OF DIFFERENTIAL PRESSURE CONTROL OF A RECIPROCATING ELECTROKINETIC PUMP,” filed May 7, 2012, incorporated by reference.

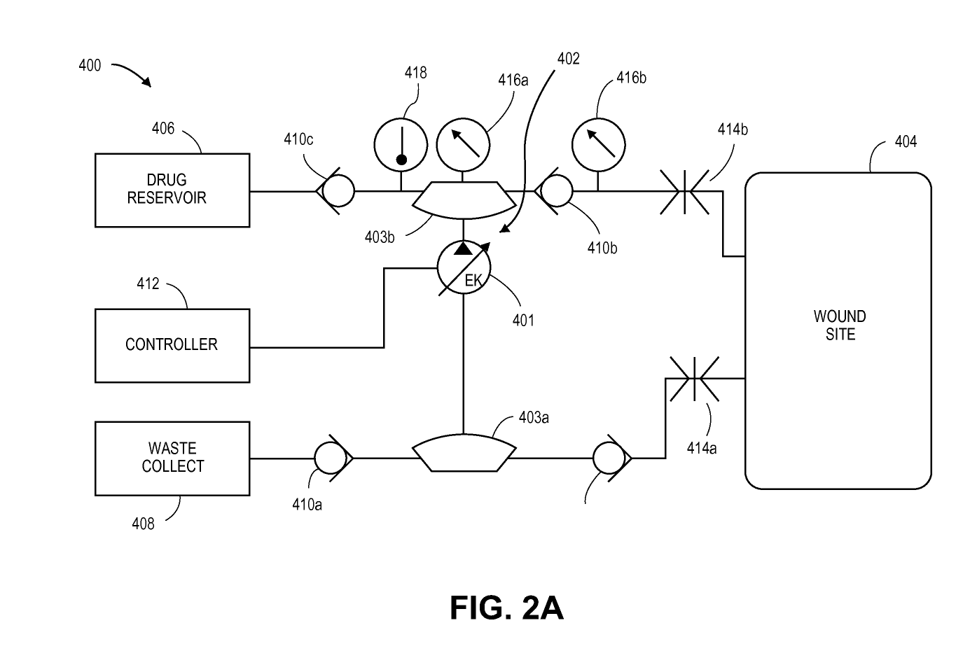

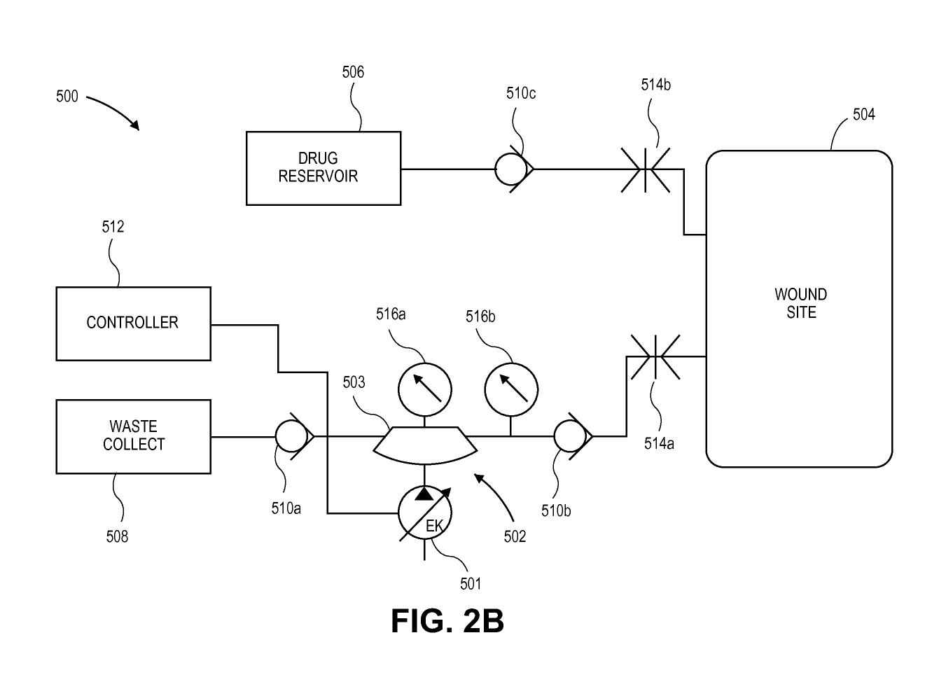

Referring to FIGS. 2A-2B, closed wound treatment systems can include a patch and a single pump system used to both deliver fluid to the patch and evacuate fluid from the patch.

For example, as shown in FIG. 2A, a wound treatment system 400 can include a single pump system 402 having a reciprocating electrokinetic engine 401 that powers both an evacuation pump 403a and a delivery pump 403b. An exemplary reciprocating electrokinetic pump is described in commonly assigned, co-pending U.S. Provisional Patent Application Ser. 61/482,960 filed on May 5, 2011 titled “System and Method of Differential Pressure Control of a Reciprocating Electrokinetic Pump.” The system 400, like system 100-300, can include a patch 404, a drug reservoir 406, a waste reservoir 408, and inlet and outlet check valves 410a,b,c,d. A single controller 412 can be used to control the activation of the electrokinetic engine 401. The system 400 can further include one or more quick disconnects 414a,b, pressure sensors 416a,b (two pressure sensors—one before and one after one of the pump systems 102—can be used rather than four if the delivery stroke and the evacuation stroke are assumed to be approximately the same), and a temperature sensor 416, similar to the wound systems described above.

As shown in FIG. 2B, a wound treatment system 500 can include a single pump system 502 (having an electrokinetic engine 501 and an electrokinetic pump 503) configured to “directly drive” both delivery of fluid to the patch 504 and evacuation of fluid from the patch 502. A single controller 512 can be used to control the electrokinetic engine 501. In this configuration, the pump system 502 can evacuate fluid from the patch 504 into the waste reservoir 508, and the negative pressure associated with doing so will result in pulling fluid from the drug reservoir 506 into the patch 504. The check valves 510a,b,c can be used to maintain the proper pressure in the pump (check valves 510a,b are used to evacuate fluid from the wound site while check valve 510c is opened once the pressure in the patch is low enough to allow fresh drug to flow into the wound site). Pressure sensors 516a,b can be used to determine the pressure and therefore the amount of drug pumped into the patch 504.

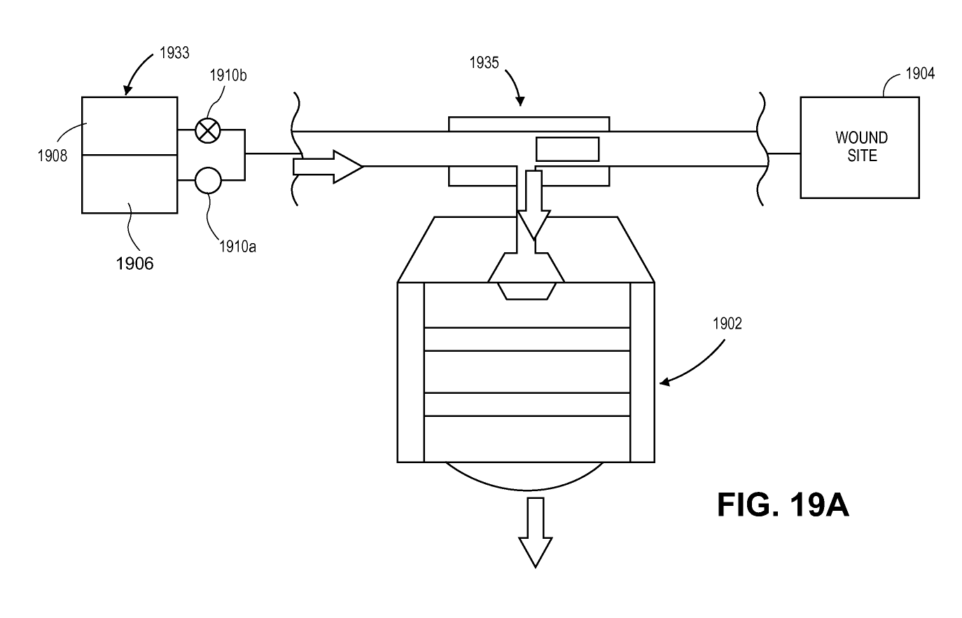

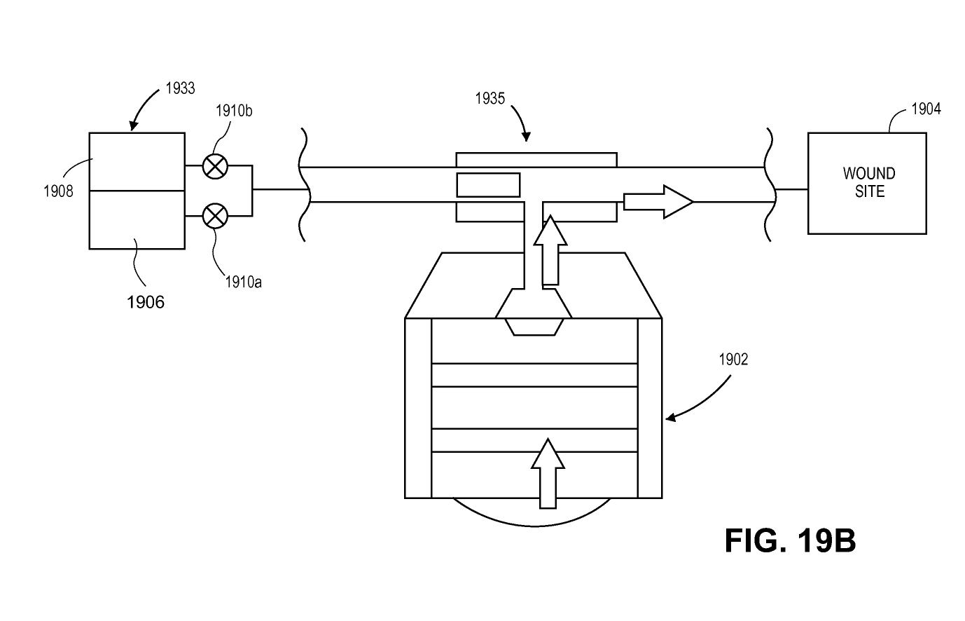

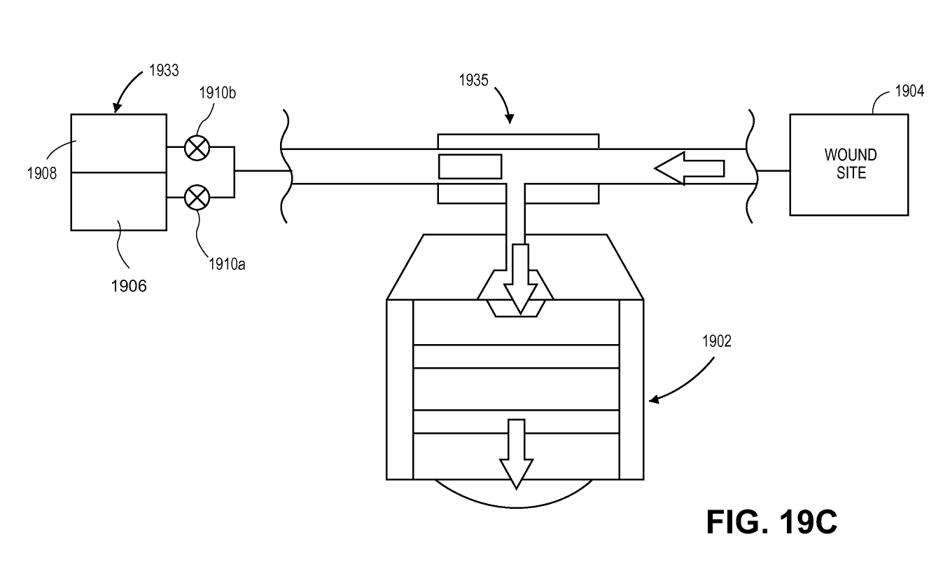

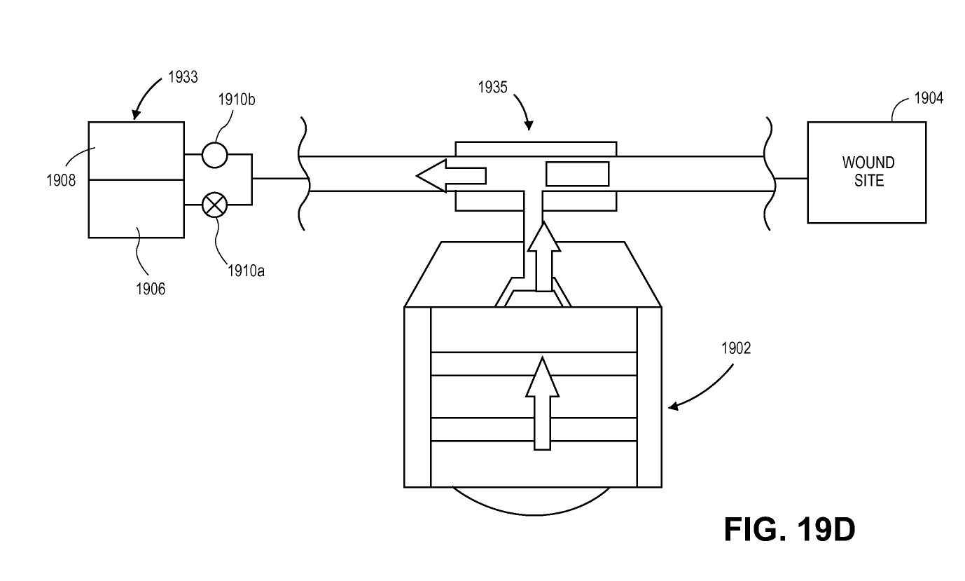

Other configurations of the wound treatment system, such as the single pump system described below with respect to FIGS. 19A-19D are possible. Further, it is to be understood that the various components of systems 100-500 can be interchanged, combined, added, or subtracted while still falling within the scope of this disclosure.

### Use of System

The components of the systems described herein can be used to perform wound treatment protocols or wound therapy to control or manipulate the environment of the wound site to facilitate healing of the wound.

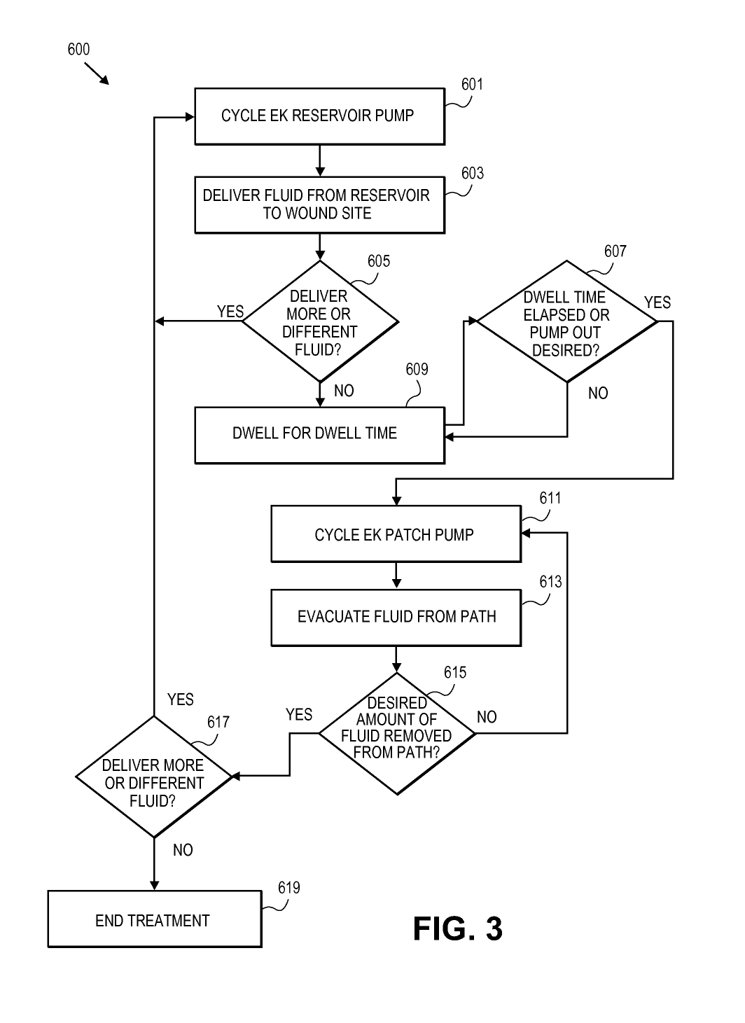

FIG. 3 is an exemplary flowchart 600 of a method of providing fluid to and from a wound patch treatment area using a wound treatment system with two pumps (e.g., systems 100-400 above). At step 601, the electrokinetic reservoir pump is cycled. At step 603, fluid from the drug reservoir is delivered to the wound site under the patch. At step 605, it can be determined whether to deliver more of the same or different fluid to the wound site. If more fluid is desired, then the cycle can begin again at step 601 (if a different fluid is desired, the patch can be disconnected from the drug reservoir through a quick disconnect mechanism and reattached to a new reservoir containing a different drug). If no more fluid is desired, then, at step 609, the system can dwell for a desired time to allow fluid to remain the wound treatment volume for a period of time, called “dwell time.” The dwell time can permit the wound to soak in the fluid, thereby cleaning or treating the wound site with the fluid. At step 607, it can be determined whether the dwell time has elapsed or whether evacuation is desired. If not, then the system can continue to dwell. If so, then at step 611, the electrokinetic evacuation pump can be cycled. At step 613, fluid from the patch can be evacuated. At step 615, it can be determined whether the desired amount of fluid has been removed from the patch. If not, then the system can continue to cycle the EK evacuation pump at step 611. If so, then at step 617, it can be determined whether more fluid should be delivered. If so, then the cycle can begin again at step 601 (if a different fluid is desired, the patch can be disconnected from the drug reservoir through a quick disconnect mechanism and reattached to a new reservoir containing a different drug). If not, then the treatment can be ended at step 619.

The systems described herein can be used to deliver saline or one or more pharmacologically active agents to assist in wound treatment. For example, the pharmacologically active agents can assist wound treatment by impeding or preventing other processes that may be occurring at the wound site, such as infection, swelling and scar formation. As another example, the pharmacologically active agents provide for irrigation or lavage of the wound treatment area or within the wound treatment volume. Exemplary pharmacologically active or inactive agents useful for one or more of the purposes described above include those agents commonly used in wet wound therapy, such as antimicrobials, antibiotics, growth factors, or wound cleansers, for example Bard Biolex Wound Cleanser, Carrington Laboratories Carraklenz cleanser, Carrington Laboratories MicroKlenz wound and skin cleanser, Coloplast Comfeel Sea-Clens, Century Pharmaceuticals Dakin's Solution, Smith & Nephew 44900 Dermal Wound Cleanser, Hollister Restore Wound Cleanser, Convatec 121222 Shur-Clens Wound Cleanser, amphotericin B, Cephalexin, ceftazidime, gentamicin, penicillin, piperacillin-tazobactam, streptomycin, or vancomycin.

In addition to the management of fluid delivery and removal from the wound treatment site, the treatment system described herein may also be used to adjust or manipulate the environment of the wound treatment area or volume. In this aspect, parameters of the treatment area or volume such pressure, temperature, humidity, moisture vapor transfer rate, or other time rate of change of environmental parameters can be used to adjust the method of providing wound therapy. In this aspect, the system controller receives wound site environmental information as an input that is used to determine whether one or more steps of a wound therapy method are to be added, modified or removed. Modifications to a wound therapy program are wide ranging and include, for example: (a) adjusting the positive or negative pressure within the wound treatment volume; (b) executing a positive pressure therapy protocol within the wound treatment volume; (c) executing a negative pressure therapy protocol within the wound treatment volume; (d) adjusting the dwell time of a particular fluid, adjusting the mixing ratio of two or more fluids; or (e) adjusting the timing of the introduction or removal of two or more fluids into the wound treatment volume. In one aspect, the pressure range used in the wound treatment volume is limited so that most of any volume change in the wound treatment volume is directed to the deformation of the patch not the patient's skin the wound, periwound or tissue within the treatment volume. In other words, the patch will deform before the pressures exerted deform tissue. However, in an alternative aspect, the controllable pressure adjustment within the wound treatment volume may be used to deflect the tissue within the treatment volume. In this manner, the pressure is increased so that the tissue within the wound treatment volume deforms in a manner to help stimulate wound repair, increase circulation, break up biofilm and promote good tissue growth. In one aspect, the patch deformation may be designed so that even with the increased pressure range the degree of tissue deformation is controlled including deformation of the patch at increased pressures without the inner walls or surfaces of the patch coming into contact with the wound or periwound region of the tissue treatment area.

### Patch Attachment to Wound

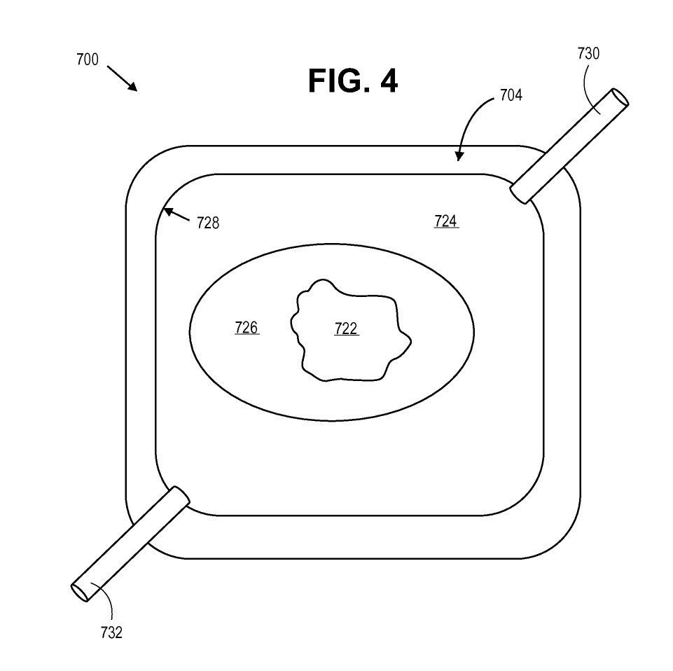

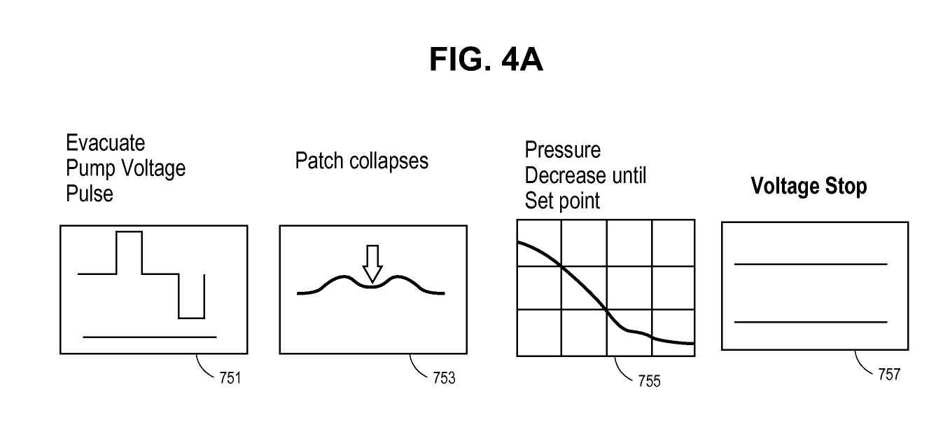

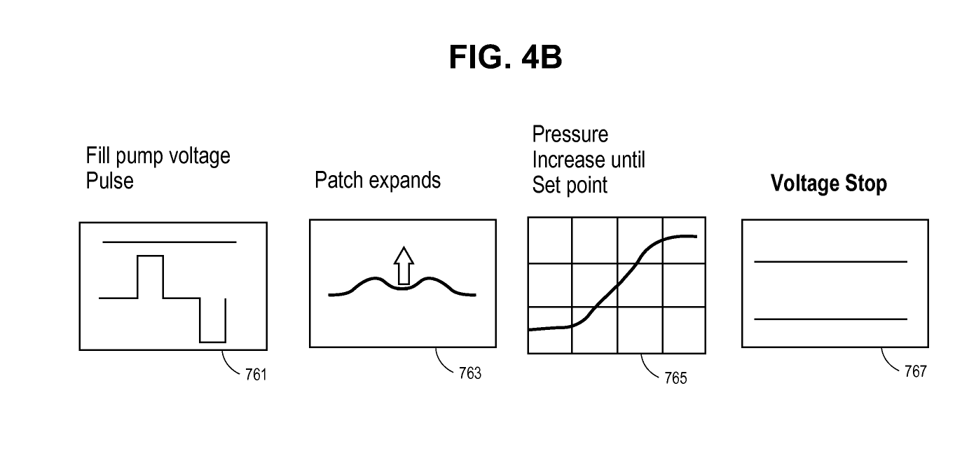

FIG. 4 is a schematic view of a closed wound treatment area 700. A treatment patch 704 can be placed around, but not in contact with a wound area 722 undergoing treatment. The treatment patch 704 can provide a fluid and pressure tight enclosure for the wound area 722. The patch 704 can adhere to the patient, forming a treatment area on the epidermis 724 of the patient. The treatment area includes the wound area 722 and periwound area 726. An inner surface of the patch ceiling can be positioned above the treatment area so that when the patch 704 is attached to the epidermis 724 of the patient, a treatment volume is created. A treatment volume is formed by the interior wall of the patch ceiling and the inner perimeter 728. As such, once the patch perimeter is attached to the epidermis around the wound treatment site, a fluid and pressure tight treatment volume is formed.

In the illustrative embodiment of FIG. 4, an inlet 730 and an outlet 732 configured to be in communication with the electrokinetic pump assembly are shown. In this configuration, the inlet 730 and outlet 732 are in the corners of the patch 704. This location in the corners helps to maintain the fluid communication thought the inlets and outlet as the wound patch walls and ceiling deform under the changing pressure conditions during therapy. In one aspect, one or both of the inlet or the outlet is located directly adjacent to the bottom of the patch perimeter. In other alternative configurations, the inlet or the outlet are placed within about 15 mm, about 10 mm or about 5 mm from the bottom of the patch. Additionally or alternatively, the inlet or outlet may be positioned on the upper walls or top surface of the patch. Additionally or alternatively, one or more ports, fittings, or sealed openings may be provided in the patch wall to permit access or connection. Such ports or openings may be used to sample the fluid or environment within the treatment volume or add or remove fluid from the treatment volume, including the injection of a pharmacologically active ingredient, tissue growth factor, wound treatment agent, engineered cell, growth factor or component used in tissue engineering or gene therapy.

Various penetrations though the patch body may be provided to allow, for example, fluid flow paths into the treatment volume for irrigation, lavage or delivery of pharmacologically active agents. Additionally, fluid flow paths into the treatment volume may be provided for use in evacuating the treatment volume or applying low pressure or even vacuum based wound therapy. Still other openings into the therapy volume may be provided to allow for instruments to sample or monitor the environment within the treatment volume. Alternatively or in addition, sampling and monitoring may be accomplished using the existing conduits or connects provided for the one or more pumps coupled to the treatment volume or other connection ports.

### Use of the Patch

FIG. 4A illustrates the result of the evacuation cycle operation on the patch and wound treatment volume. As shown in the graph 751, the system controller can apply a voltage to the evacuation pump. As shown at 753, the patch begins to collapse as voltage is applied to the evacuation pump because, as shown at 755, the pressure within the wound treatment volume decreases. This process continues until a control set point is reached. The set point may be based on any appropriate control variable. For example, the set point may be a time limit, a pressure reading, or a combination of variables. Once the set point is reached, as shown at 757, the drive signal to the evacuation pump ceases.

FIG. 4B illustrates the result of the delivery cycle operation on the patch and wound treatment volume. As shown in the graph 761, the system controller can apply voltage to the delivery pump. As a result, the patch begins to expand (as shown at 763) and the pressure within the wound treatment volume increases (as shown at 765). This process continues until a control set point is reached. The set point may be based on any appropriate control variable. For example, the set point may be a time limit, a pressure reading, or a combination of variables. As shown at 767, once the set point is reached the drive signal to the delivery pump ceases.

The patches herein can advantageously be used to perform wound therapy without removing the patch. For example, fluid can be delivered and evacuated repetitively without removing the patch. Indeed, a second fluid different from the first can be delivered to the wound area without removing the patch. In some embodiments, the patch can remain in place during a wound protocol for at least 24 hours, such as at least 48 hours, for example at least 72 hours.

### Patch Characteristics

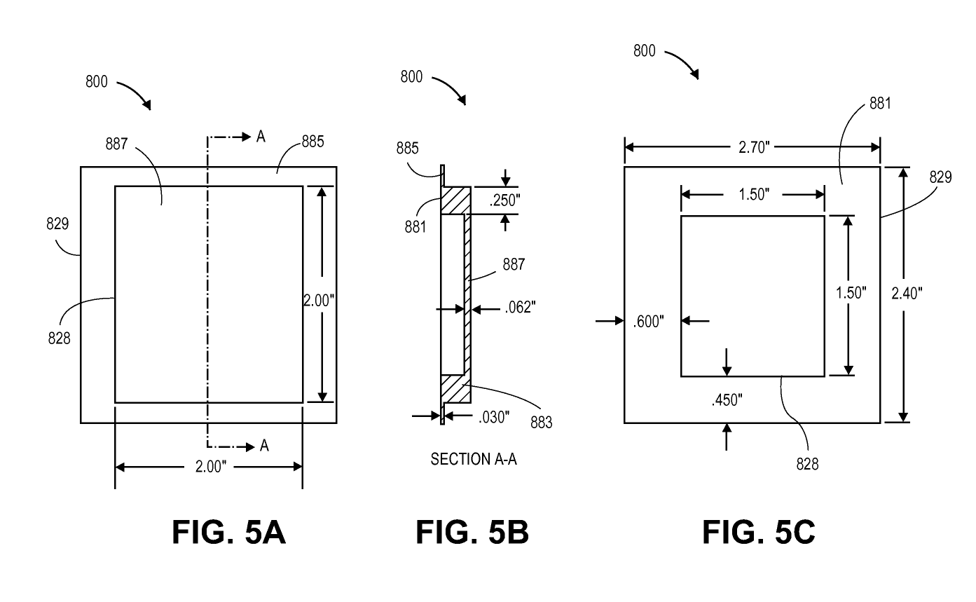

Various patches that can be used with the wound treatment systems described herein are described with respect to FIGS. 5-14. It is to be understood that the components of various patches can be interchanged, replaced, or used with any other patch described herein.

FIGS. 5A, 5B and 5C are top, section view and bottom up view, respectively, of an exemplary flat top wound patch 800. The flat top wound patch 800 includes an inner rectangular perimeter 828 and an outer rectangular perimeter 829 defining an adhesive surface 881 therebetween. The side walls 883 can extend approximately perpendicular to the adhesive surface 881, and the top surface 887 of the patch can extend approximately perpendicular to the walls 883, thereby forming a flat top. Further, the bottom of the patch can include a lip 885 extending around the outer perimeter 881 to provide extra area for adhesion. The dimensions of the flat top wound patch 800 can vary. In one specific embodiment, the adhesive surface can be about 0.450 inches on one side and 0.600 inches along the other. The exterior perimeter 829 dimensions of the patch can be, for example, 2.4 inches by 2.7 inches. The lip 885 can be approximately 0.030 inches thick. The lower surface of the adhesive area 881 can be covered with a suitable adhesive, such as a water-proof pressure sensitive adhesive or other biocompatible adhesive suited to maintaining the patch on the epidermis during the wound treatment, e.g., silicone adhesive. In this illustrative embodiment, the wound treatment area is approximately a square having a side length of 1.5 inches. The treatment volume can be 8.8 ml. The ratio of treatment volume to treatment area for this illustrative embodiment is 0.6 ml/cm2.

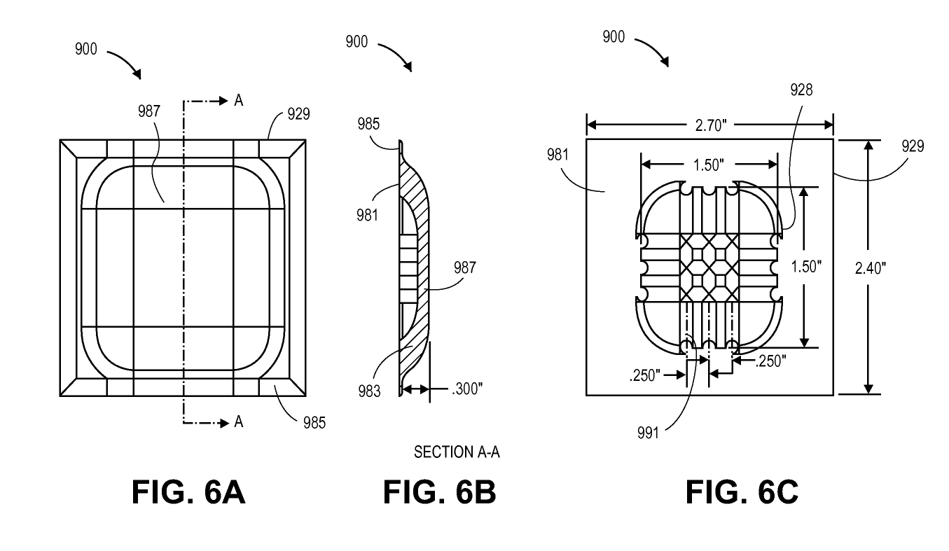

FIGS. 6A, 6B and 6C are top, section, and bottom views, respectively, of an exemplary rounded top wound patch 900 having internal reinforcement elements. The rounded top wound patch includes an inner perimeter 928 and an outer perimeter 929 defining an adhesive surface 981 therebetween. The side walls 983 can slope upwards to form a rounded profile up to the top surface 987. The top 987 can have rounded corners, i.e., be shaped as a square with rounded corners or an oval with flattened sides. A lip 985 can extend from the sidewalls to provide extra adhesive surface. The dimensions of the rounded top wound patch 900 can vary. In one specific embodiment, the patch has a base of 2.4 inches by 2.7 inches. The adhesive area is approximately 0.3 inches thick. The surface is covered with a suitable pressure sensitive adhesive or other biocompatible adhesive suited to maintaining the patch on the epidermis during the wound treatment. In this illustrative embodiment, the wound treatment area is has a generally rectangular base with sides having lengths of 2.7 inches and 2.4 inches. As best seen in FIG. 6B, the treatment volume is 5 ml with a 1.5 inch×1.5 inch treatment area. The ratio of treatment volume to treatment area for this illustrative embodiment is 0.34 ml/cm2.

Reinforcement elements 991 or ribs can extend throughout the inside of the patch, for example forming a crossed pattern on an inner surface of the top 987. In one embodiment, the reinforcement elements 991 can be formed of the same material as the patch 900, but be thicker than the rest of the patch. For example, the reinforcement elements 991 extend from the bottom of one side, along the ceiling of the patch, across the middle portion of the ceiling and to the bottom of the opposite side. Two pairs of three reinforcement members each can intersect at a 90 degree angle in the patch ceiling. In this illustrative embodiment, each reinforcement member 991 has a generally cylindrical shape with a radius of about 0.07 inches. The three reinforcement elements 991 are spaced about 0.25 inches apart on center.

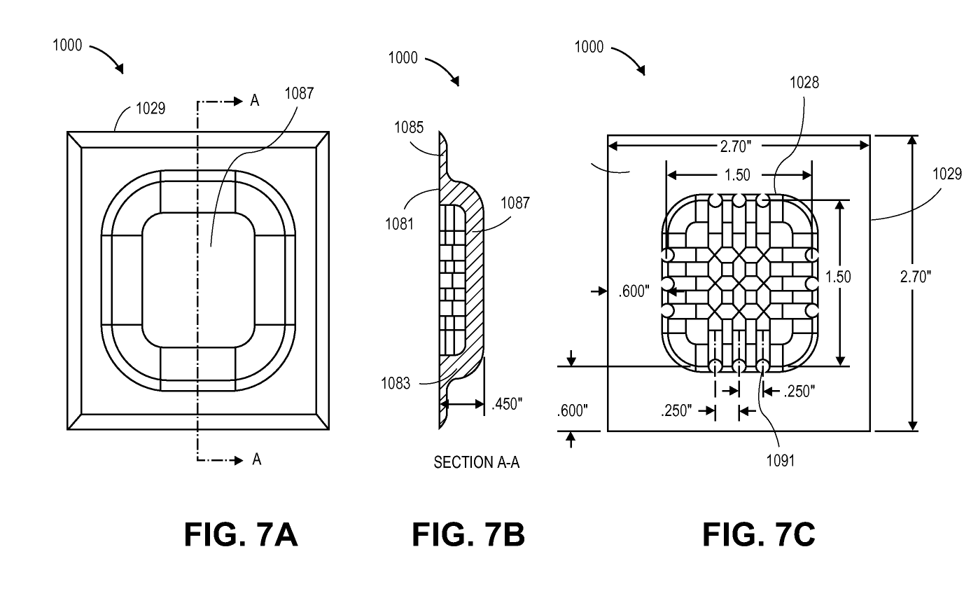

FIGS. 7A, 7B and 7C are top, section, and bottom views, respectively, of an exemplary rounded top wound patch 1000 having internal reinforcement elements. The rounded top wound patch 1000 includes an inner perimeter 1028 and an outer perimeter 1029 defining an adhesive surface 1081 therebetween. The adhesive surface 1081 is covered with a suitable pressure sensitive adhesive or other biocompatible adhesive suited to maintaining the patch on the epidermis during the wound treatment. In this illustrative embodiment, the patch has a square base and a flattened oval or rounded square top 1087. A ledge 1085 can extend from the sidewalls 1085 to provide extra adhesive area. The overall dimensions of the rounded top wound patch 1000 can vary. In one specific embodiment, the adhesive surface 1081 is about 0.6 inches on one side and 0.6 inches along the other. The square base can be 2.7 inches on a side with a height of about 0.450 inches. The treatment volume can be 10 ml with a 1.5 inch×1.5 inch treatment area. The ratio of treatment volume to treatment area for this illustrative embodiment is 0.689 ml/cm2.

Similar to the wound patch 900, the wound patch 1000 (or any of the patches described herein) can include reinforcement members 1091 extending throughout the inside of the patch, such as ribs of thicker material. The ribs can extend along the patch in a variety of patterns to provide the required support. For example, the reinforcement elements can extend from the bottom of one side, along the ceiling of the patch, across the middle portion of the ceiling and to the bottom of the opposite side. Two pairs of three reinforcement members each are shown intersecting at a 90 degree angle in the patch ceiling. In the illustrative patch 1000, each reinforcement member has a generally cylindrical shape with a radius of about 0.07 inches. The three reinforcement elements are spaced about 0.25 inches apart on center.

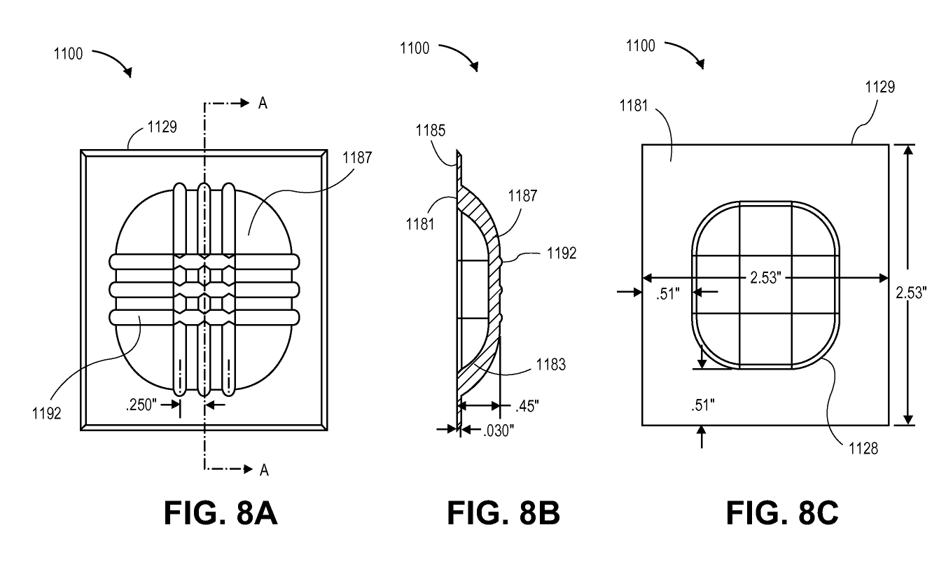

FIGS. 8A, 8B and 8C are top, section, and bottom up view, respectively, of an exemplary rounded top wound patch 1100 having external reinforcement elements. The rounded top wound patch includes an inner perimeter 1128 and an outer perimeter 1129 defining an adhesive surface 1181 therebetween. The adhesive surface 1181 is covered with a suitable pressure sensitive adhesive or other biocompatible adhesive suited to maintaining the patch on the epidermis during the wound treatment. In this illustrative embodiment, the patch has a square base and a flattened oval or rounded square top 1187. A ledge 1185 can extend from the sidewalls 1185 to provide extra adhesive area. The overall dimensions of the rounded top wound patch 1000 can vary. In one specific embodiment, the adhesive area 1181 is about 0.51 inches on one side and 0.51 inches along the other. The square base can be 2.53 inches on a side with a height of about 0.450 inches. The adhesive area is approximately 0.03 inches thick. The treatment volume can be 10 ml with a 1.5 inch×1.5 inch treatment area. The ratio of treatment volume to treatment area for the illustrative embodiment of the patch 1100 is 0.689 ml/cm2.

In contrast to the embodiments of FIGS. 6 and 7, the patch 1100 illustrated in FIG. 8 provides externally positioned reinforcement members 1192, i.e., positioned along the outer surface of the patch 1100. The reinforcement members 1192 can form a variety of patterns throughout the patch. For example, in the embodiment shown in FIG. 8, the reinforcement elements 1192 extend from the bottom of one side, along the outer surface of the patch, across the middle portion of the ceiling and to the bottom of the opposite side. Two pairs of three reinforcement members can intersect at a 90 degree angle in the patch ceiling. In this illustrative embodiment, each reinforcement member has a generally cylindrical shape with a radius of about 0.06 inches. The three reinforcement elements are spaced about 0.25 inches apart on center. The placement of the reinforcement elements on the exterior surface of the patch can provide a smooth interior surface to the treatment volume. These externally placed reinforcement members 1192 can be used with any of the patches described herein.

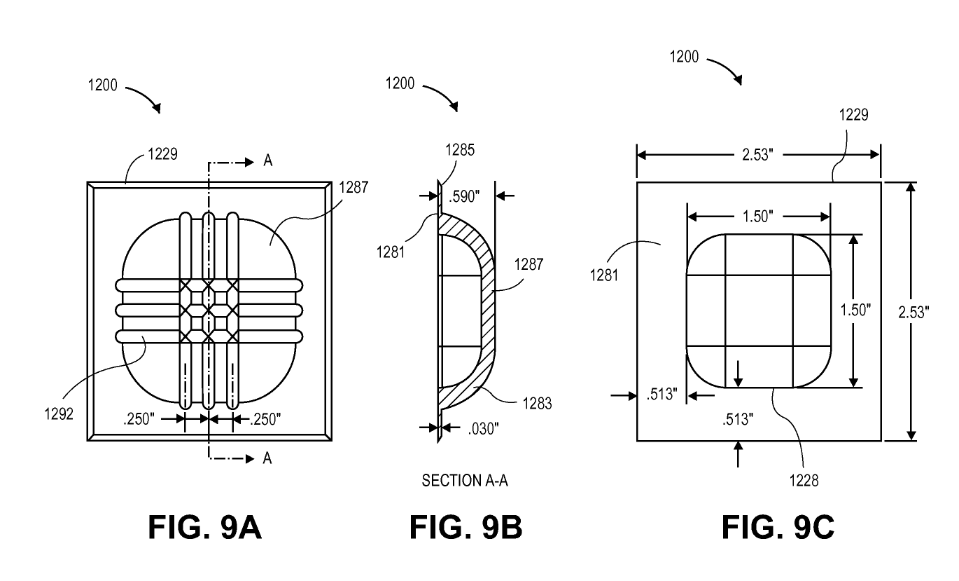

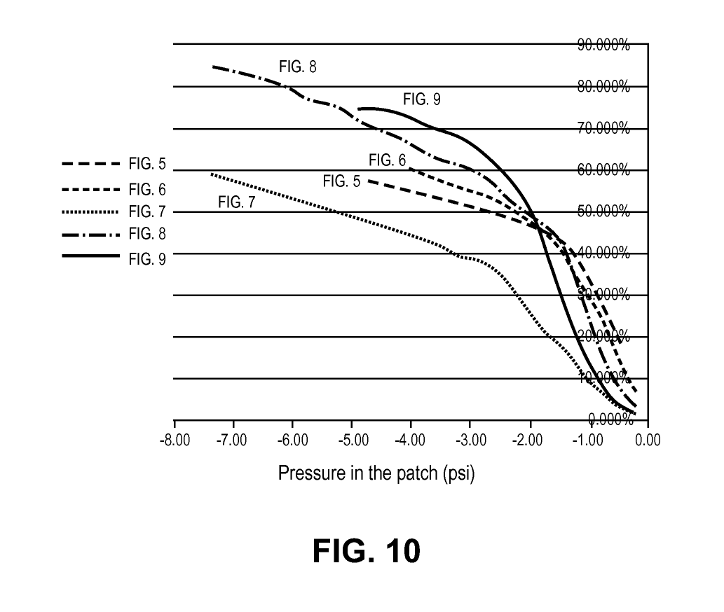

FIGS. 9A, 9B and 9C are top, section, and bottom views, respectively, of an exemplary rounded top wound patch 1200 having external reinforcement elements. The rounded top wound patch includes an inner perimeter 1228 and an outer perimeter 1229 defining an adhesive surface 1281 therebetween. The adhesive surface 1281 is covered with a suitable pressure sensitive adhesive or other biocompatible adhesive suited to maintaining the patch on the epidermis during the wound treatment. In this illustrative embodiment, the patch has a square base and a flattened oval or rounded square top 1187. A ledge 1185 can extend from the sidewalls 1185 to provide extra adhesive area. The overall dimensions of the rounded top wound patch 1000 can vary. In one specific embodiment, the adhesive area 1281 is about 0.513 inches on one side and 0.513 inches along the other. The square base is 2.53 inches on a side with a height of about 0.59 inches. The adhesive area 1281 is approximately 0.03 inches thick. The treatment volume can be 15 ml with a 1.5 inch×1.5 inch treatment area. The ratio of treatment volume to treatment area for this illustrative embodiment is 1.03 ml/cm2.

In contrast to the embodiments of FIGS. 6 and 7 and similar to FIG. 8, the patch illustrated in FIG. 9 provides externally positioned reinforcement members 1292. In this illustrative embodiment, each reinforcement member 1292 has a generally cylindrical shape with a radius of about 0.06 inches. The reinforcement elements 1192 can form a variety of patterns. For example, the reinforcement elements 1292 can extend from the bottom of one side, along the outer surface of the patch, across the middle portion of the ceiling and to the bottom of the opposite side. Two pairs of three reinforcement members each can intersect at a 90 degree angle in the patch ceiling. The three reinforcement elements are spaced about 0.25 inches apart on center. The placement of the reinforcement elements on the exterior surface of the patch provides a smooth interior surface to the treatment volume.

FIG. 10 shows a curve illustrating the percentage volume removed from the patch interior volume as a function of vacuum applied to the patch for the patch embodiments of FIGS. 5-9 made with a material having a durometer of 30 shoreA. During this test, the patch treatment volume was filled with water and then exposed to an increasing negative pressure or vacuum measured in pounds per square inch (psi). The patch of FIG. 5 had about 50% volume removed at −2 psi and did not achieve 60% volume removal even at −5 psi. The patch of FIG. 6 had about 50% volume removed at about −2.25 psi and achieved 60% volume removed at about −4.0 psi. The patch of FIG. 7 had about 50% volume removed at about −5.25 psi and is estimated that 60% volume removed would require more than −7.0 psi. The patch of FIG. 8 had about 50% volume removed at less than about −2 psi, achieved 60% volume removed at about −3.0 psi, and achieved 70% volume removed at about −4.5 psi. The patch of FIG. 9 had about 50% volume removed at less than about −1.5 psi, achieved 60% volume removed at about −2.5 psi, and achieved 70% volume removed at about −3 psi.

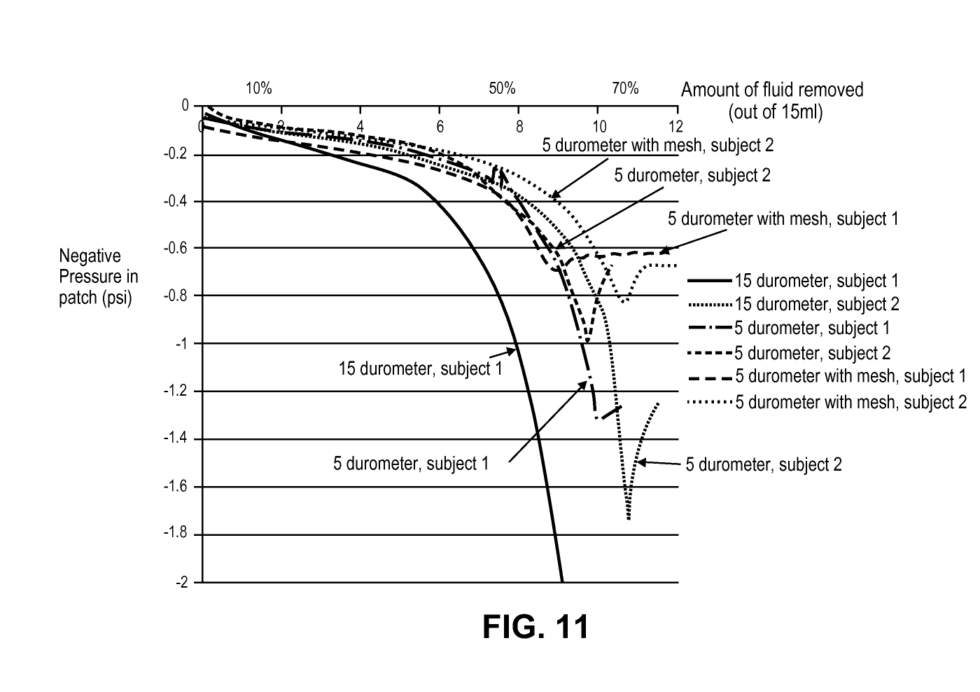

The durometer of the material for the patches described herein can be between 10 and 50 shoreA, such as between 5 and 30 shoreA, for example about 15 shoreA. The patch material can be, for example, silicone. FIG. 11 shows a curve illustrating the percentage volume removed from the patch interior volume as a function of vacuum applied to a patch having a durometer of 15 shoreA and worn by two different test subjects, to a patch having a durometer of 5 shoreA and worn by two different test subjects, and to a patch having a durometer of 5 shoreA having a mesh reinforcement member and worn by two different test subjects. Advantageously, by using a durometer of less than 30 shoreA, such as 15 shoreA, less negative pressure is required to remove 70% of fluid, resulting in greater comfort for the patient. Further, by using a patch having a mesh reinforcement, less negative pressure is required to remove 70% of the fluid.

In one aspect, patch characteristics are selected so that 60% of the wound treatment volume (i.e. the volume within the confines of the interior wall of the patch) is removed when the volume is exposed to −2 psi pressure. In another aspect, patch characteristics are selected so that 70% of the wound treatment volume is removed when exposed to −1 psi. In other embodiments, more than 70% of the fluid can be removed, such as 80-90%. At pressures above these levels, human skin may begin to deform or be damaged. Similarly on the positive pressure or supply side of patch operation, human skin begins to deform at about +1 psi. In one aspect, the patch is adapted and configured to operate within a pressure range that does not deform human tissue within the wound treatment volume.

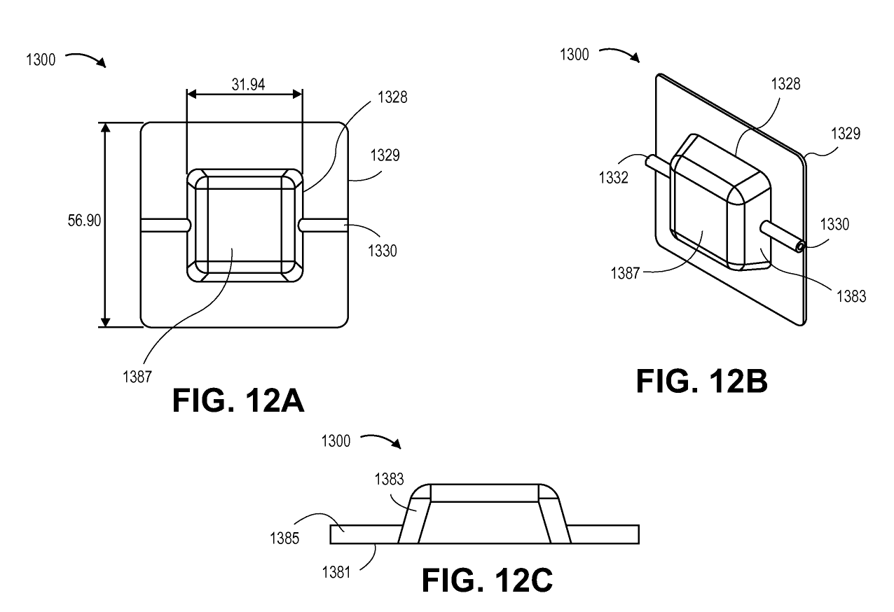

Patch configurations other than those described with respect to FIGS. 5-9 are possible. For example, referring to FIGS. 12A-12C, a simple patch 1300 can be used. The patch 1300 can have a square base with an outer perimeter 1329 and an inner perimeter 1328. The patch can further have an approximately square top surface 1387 with slightly rounded corners. The side walls 1383 can tilt inwards slightly from the base to the top surface 1387. Further, a lip 1385 can extend from the sidewalls 1383 to create a larger adhesive surface (the adhesive surface 1381 extends along the bottom of the patch). The patch 1300 can have an inlet 1330 and an outlet 1332 on opposite side walls. In one embodiment, the patch 1300 is formed of polyethylene, such as two layers of polyethylene film. The two layers of polyethylene film can be adhered together, such as hot melted together. In one embodiment, the polyethylene films are melted together around tubes forming the inlet 1330 and the outlet 1332 to capture the tubes therebetween. In one specific embodiment, the simple patch 1300 has no reinforcement members. The simple patch 1300 can have a stiffness such that there is no major deformation of the patch at pressures of less than 0.2 psi, such as less than 0.1 psi. The dimensions of the simple patch 1300 can vary. In one specific embodiment, the base is approximately a square with dimensions of 56.9 mm on each side while the top surface 1387 is approximately a square with dimensions of 31.94 mm on each side. The simple patch 1300 can advantageously provide enough stiffness to both hold fluids and be evacuated without touching the skin of the patient.

In some embodiments, a protective shell, such as a vacuum formed shell made of a plastic, such as PETG and/or a foam support can be used to sit over and protect one or more of the patches described herein, such as the simple patch 1300. The protective shell can guard against accidental emptying of fluid from the wound site caused by bumping of the patch during ordinary wear and use.

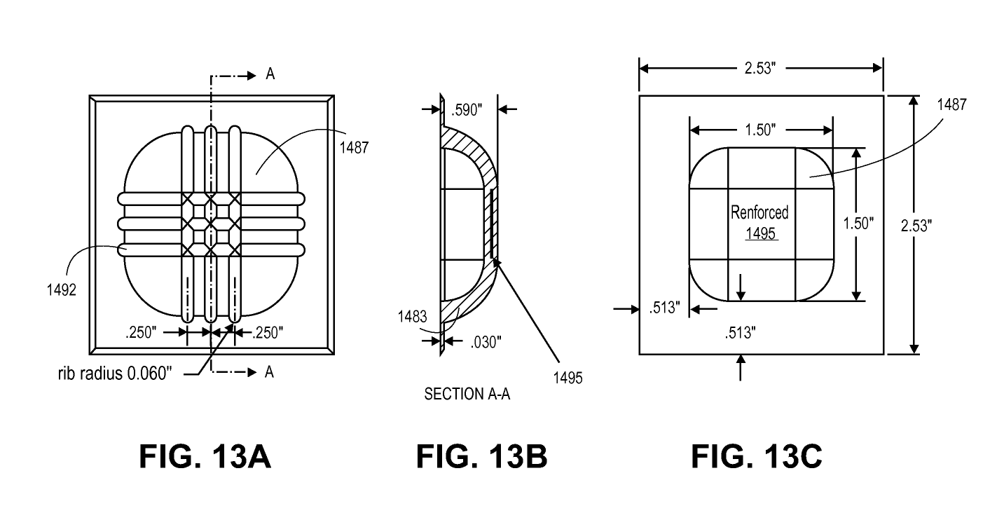

In some embodiments, the reinforcement elements for the patches described herein are not of the same composition or material, but instead can be selected to the reinforcement parameters of the area or portion of the patch to which it is attached. For example, as shown in FIGS. 13A-13C, a reinforcement element 1495 may be added along the top wall of a patch 1400 so that, as the walls 1483 of the patch deform, the top 1487 remains nearly flat. For example, the reinforcement element 1495 can be approximately the same shape as the top surface of the patch, such as square. Such reinforcement of the upper surface of the wound treatment volume is believed to assist in preventing the interior top surface portion of the wound volume from contacting the wound during pressure changes. The reinforcement element 1495 can be made of a material that provides added stiffness to the patch. In some embodiments, the mesh is a metal mesh or a nylon mesh. The mesh can include 0.03 inch diameter wire and 0.1 inch spacing between the wires. In some embodiments, the mesh can be cut to about 1 inch by 1 inch. Such reinforcement of the upper surface of the wound treatment volume advantageously provides for substantially constant deformation across the patch to provide more consistent evacuation and flushing and the wound site and assists in preventing the interior top surface portion of the wound volume from contacting the wound during pressure changes. A reinforcing element may be made of the same or different material used to fabricate the patch. The reinforcement element can be used with or without additional reinforcement members. For example, the patch 1400 shows external reinforcement members 1492 extending in conjunction with the reinforcement element 1495.

The dimensions of the patch 1400 can vary. In one specific embodiment, the base is approximately a square with dimensions of 2.53 inches per side while the top surface is a rounded square with dimensions of approximately 1.5 inches on each side. The adhesive area can have a width of approximately 5.13 inches while the ledge can have a height of 0.590 inches. The reinforcement members 1492 can have a radius of approximately 0.060 inches, and the members 1492 can be located approximately 0.060 inches apart.

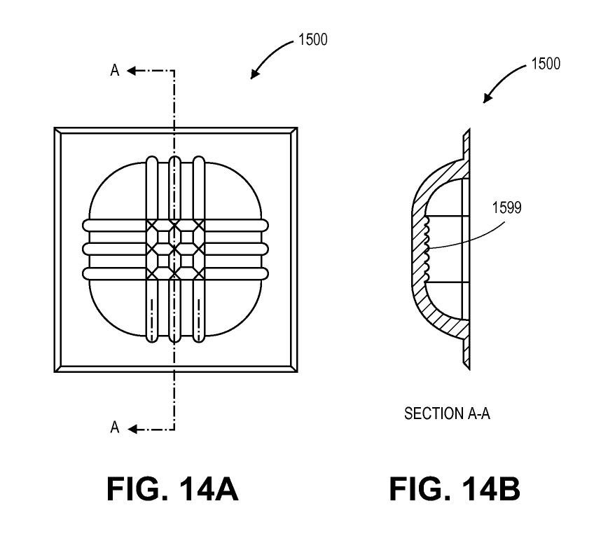

In some embodiments, raised portions or bumps can be placed on the underside of the patch. For example, as shown in FIGS. 14A-14B, raised portions or bumps 1599 are provided on the underside of the patch 1500. The bumps can be made of the same materials as the rest of the patch or of a different material. In some embodiments, the bumps can be arranged in a grid, such as 7 bumps×7 bumps. Further, the bumps can be hemispheres, such as hemispheres of 0.08 inches in diameter. The bumps may be slightly pointed, i.e. have a tip rather than a rounded surface. Advantageously, the bumps can break up the surface area of the underside of the patch to prevent the bottom surface from suctioning to the wound. In some embodiments, the bumps can also advantageously slightly contact the wound to stimulate a healing response.

In some embodiments, a patch may include one or more windows or view ports to permit visual observation of the wound treatment volume or of the periwound, wound and/or epidermis regions. The window or view port may be provided anywhere on the patch that permits observation of the wound treatment volume during therapy. The window may be located, for example, on a sidewall, on or near an upper surface or roof of a patch or along a rim or peripheral portion. Other locations are possible depending upon the specific wound location and patch placement and geometry.

In some embodiments, the adhesive area (i.e., the width of the material between Pi and Po) ranges from about ⅜ inch to about ½ inch. One suitable biocompatible adhesive is a medical grade silicone adhesive. One commercially available adhesive is Hollister 770 stray on adhesive.

In some embodiments, the non-contact patch may be reinforced using techniques other than those illustrated and described above in FIGS. 5A-9C. One or more reinforcing elements may be embedded within or attached along all or a portion of a wall of a patch. Exemplary reinforcing elements may come in a variety of different shapes including, for example, wire, mesh or strips. Exemplary reinforcing materials include nitinol, carbon fiber and metals. The reinforcement element may be in one or more locations on, in or within the patch.

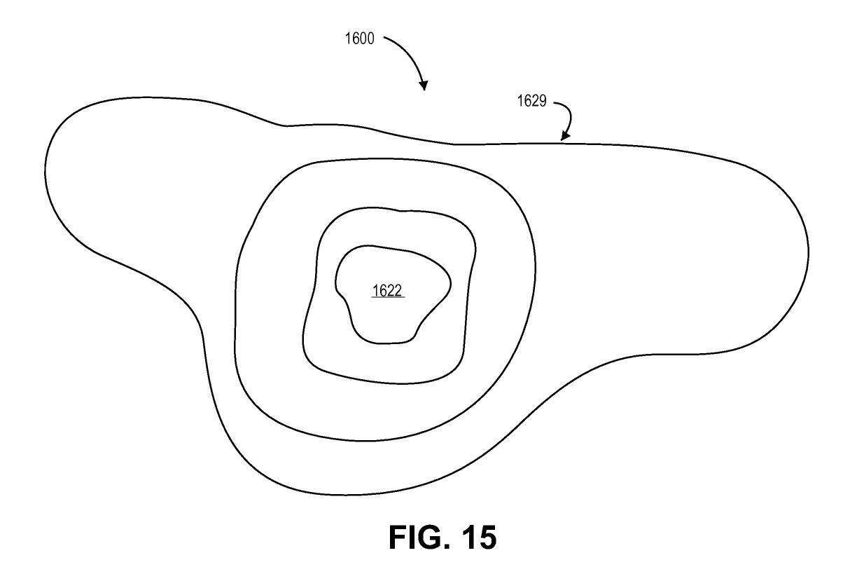

In some embodiments, the patches described herein can have dissimilar inner and outer perimeters and/or uneven forms. FIG. 15 is a top down view of a patch 1600 having dissimilar inner and outer perimeters in place over a wound 1622. In this embodiment, the outer perimeter 1629 has elongated sides similar to straps that may be used to affix the patch 1600 to a wound treatment site by wrapping around a limb being treated, for example. Other shapes and sizes of patches may be provided depending upon the specific topography of the wound treatment site, wound size and shape as well as the size and shape of the impacted periwound site.

### Test Component

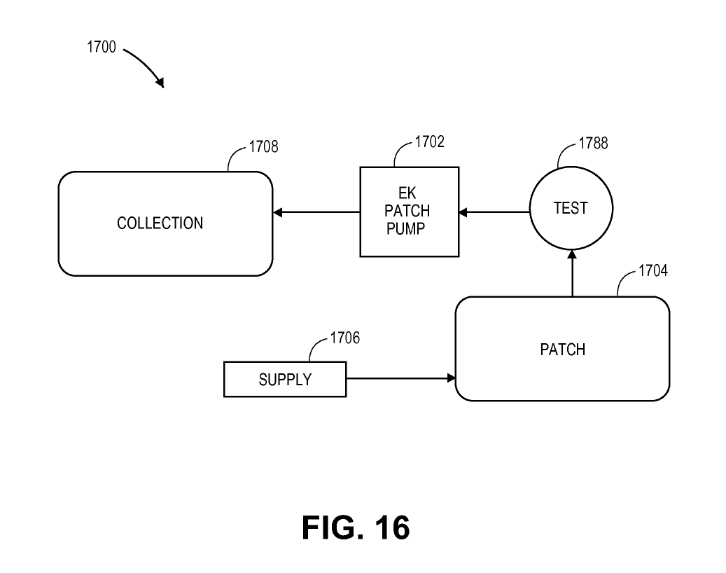

FIG. 16 is a schematic view of an electrokinetic pump powered wound treatment system 1700 that includes a test component 1788. In this illustrative embodiment, the test component 1788 is positioned between the waste reservoir 1708 and the patch 1704, such as between the evacuation pump 1702 and the patch 1704. Thus, on one embodiment, the test component is configured as an exudate sampler. Although only a single pump system 1702 is shown in FIG. 16, other configurations are possible (such as a configuration including a separate evacuation pump and delivery pump). In another aspect, the test component may be on the outlet of the pump or taken from the collection reservoir. In other additional aspects, the test component can be configured as a sample draw connection for a manual test or for introducing the sample to a remote testing device.

The test component 1788 can collect samples of fluid, such as fluid removed from the wound site. Treatment volume fluid testing can then be conducted on the liquids removed from the system. Liquids from the wound treatment system can be analyzed, for example, to determine the contents of the sampled volume, thereby determining the effectiveness of a specific treatment or therapeutic agent.

In one aspect, the results produced by the testing component are used as feedback into the wound therapy control system. Based on feedback from the test component, the wound therapy control system may adjust one or more parameters of the wound care therapy regime such as positive pressure applied to or the time rate of change of the positive pressure applied to the wound treatment volume, negative pressure applied to or the time rate of change of the negative pressure in the wound treatment volume, the dwell time of a particular fluid provided into the treatment volume, the removal rate or the injection rate of a fluid to the wound treatment volume, and the like.

### Divided Container for Supply/Collection

Any of the wound care systems described herein can be used with a divided container that includes both the waste reservoir and the drug or treatment reservoir.

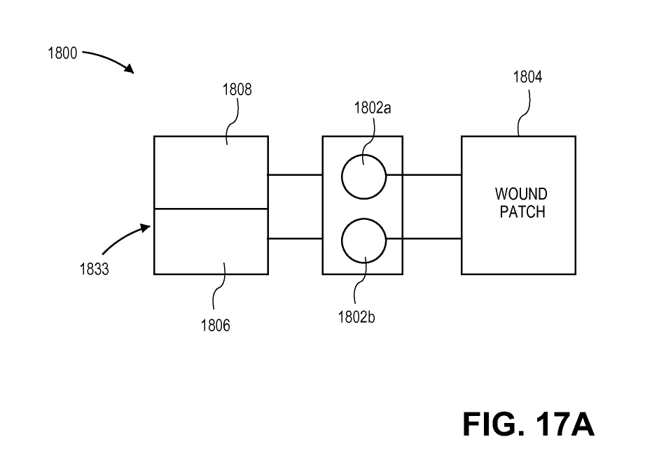

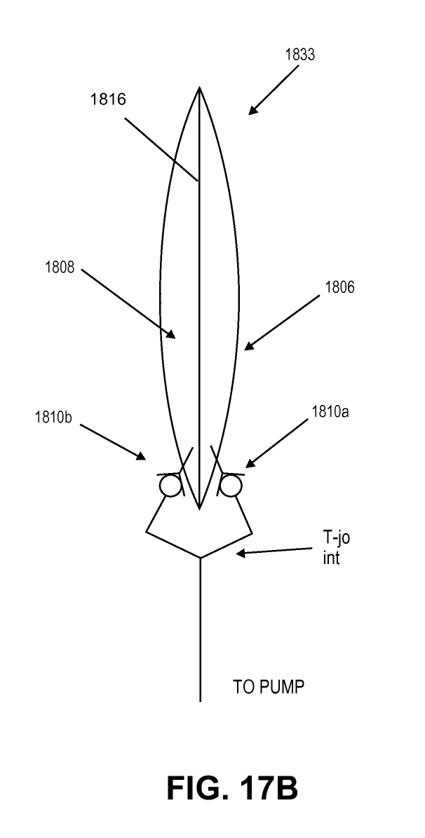

For example, FIG. 17A is a schematic view of a closed patch wound treatment system 1800 having a divided container 1833 that houses both the supply reservoir 1806 and the waste reservoir 1808. The waste reservoir can be connected to an evacuation pump system 1802a while the supply reservoir can be connected to a supply pump system 1802b. The pump systems 1802a,b can in turn be fluidically connected to the wound area under the patch 1804.

FIG. 17B is an enlarged view of the divided container 1833 of FIG. 17A. The drug and collection container 1833 is a double lumen container. The container 1833 includes a drug chamber 1806 and a collection chamber 1808. The drug chamber 1806 can contain a liquid therapeutic agent for delivery to the wound site. The collection chamber 1808 can be initially empty. Check valves 1810a,b can be used to only draw from one chamber and only deliver to the other chamber. In the illustrated configuration, the drug chamber check valve 1810a only allows flow from the drug chamber 1806. The collection chamber check valve 1810b only allows flow into the collection chamber 1808. In aspect, the drug and collection container 1833 has a total volume of from about 250 ml to 1 liter or larger, depending upon drug regime and evacuation protocol. It is to be appreciated that the drug and collection container 1833 may be scaled to pending upon the particular wound therapy procedure undertaken.

The drug and collection containers can be separated by a movable member 1816. The movable member 1816 can be configured such that the size of the respective chambers 1808, 1806 can change depending on which chamber is the fullest. That is, the movable member 1816 can be moved such that, at the beginning of the wound protocol, the drug reservoir 1806 fills substantially all of the container 1833 while the waste reservoir 1808 fills little to none of the container 1833. As fluid is delivered from the reservoir 1806 and pumped into the reservoir 1808, the movable member can move, allowing the size of the waste reservoir 1808 to increase and the size of the drug reservoir 1806 to decrease. The movable member can be, for example, a thin plastic film. In one embodiment, the total volume of the container 1833 is approximately 250 ml, allowing for 250 ml of drug to be delivered and allowing for 250 ml of waste to be collected as the movable member moves.

One advantage of using a combination drug and collection chamber is that it provides a more efficient connection of the chambers to the EK pump, valve and piping. The use of check valves minimizes the work needed by the doctors and nurses who no longer have to a change dressing. Instead, the EK pump and valve controls described herein maintain the wound treatment volume according to the wound therapy protocol. A healthcare provider need only replace the drug chamber once it is empty and/or when the extraction chamber is full. In one aspect, the drug and collection container is connected using a luer connection. In still other aspects, the wound therapy control system monitors or is programmed to calculate the stoke volume and number of strokes taken by the EK engine. Based on EK engine pump parameters and performance information, the wound therapy control system may predict, estimate or provide a warning when the container may require service.

Multiple reservoir systems using electrokinetic pumps may also be configured such as those described in commonly assigned U.S. Pat. No. 7,517,440 filed Apr. 21, 2005, incorporated herein by reference.

System with Single Electrokinetic Pump

As described above with respect to FIGS. 2A and 2B, in some embodiments, a single pump can be used to both evacuate and pump fluid into the patch.

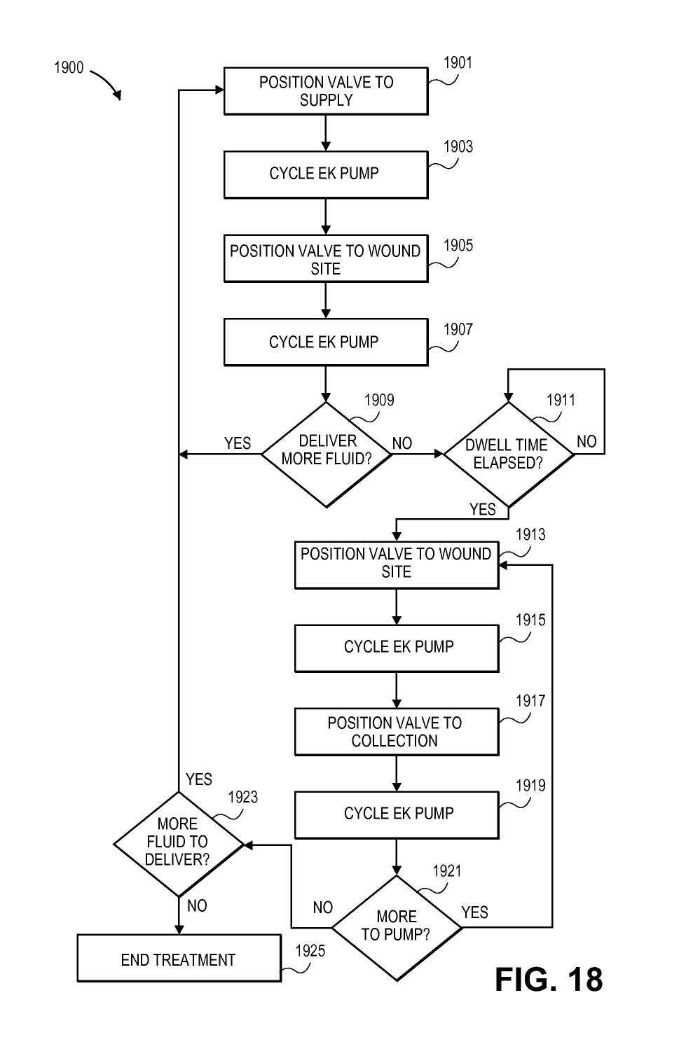

In one embodiment, as described with respect to the flow chart 1900 of FIG. 18 and FIGS. 19A-D, a three-way valve 1935 connects a drug and collection container 1933 to the wound site under the patch 1904 and an electrokinetic pump system 1902. A T-connection 1937 and pair of check valves 1910a,b isolate the drug reservoir 1906 and collection reservoir 1908 within the drug and collection container 1933. The valve 1935 may be a solenoid or a magnetic latch type. By changing the order the solenoid valve opens and closes, fluid in the wound care circuit may be moved using the same pump 1902.

FIG. 18 is a flow chart 1900 for providing therapy to a closed wound treatment site using a single electrokinetic pump system as shown in FIG. 19A-19D. FIGS. 19A-19D illustrate the valve configurations corresponding to the method described in FIG. 18.

At step 1901, the T-valve is positioned to supply, and at step 1903, the EK pump is cycled. As shown in FIG. 19A, the check valve 1910a is set to open to drug chamber as the EK engine 1902 strokes to pull fluid to the rear EK diaphragm (as shown by the arrows in FIG. 16). As a result, fluid flows from the drug chamber 1906 through the open T-valve 1935 and into the pump chamber of the electrokinetic pump system 1902. The closed check valve 1910b in the collection chamber side prevents the fluid from coming out.

At step 1905, the T-valve is positioned to wound site. As shown in FIG. 19B, the T-valve is open for delivery. At step 1907, the EK pump cycles the fluid to deliver fluid from the pump system 1902 to the wound patch 1904.

The next step in the flowchart 1900 is to determine whether more fluid is to be delivered to the wound site at step 1909. If “yes” then the process of cycling the pump to deliver fluid to the wound site continues at step 1901 until the desired volume of fluid is delivered.

If all fluid has been delivered, then at step 1911 it is determined whether the fluid in the wound treatment volume should remain or be removed. The duration of fluid within the wound treatment volume is referred to herein as dwell time. Additionally or alternatively, the wound therapy protocol may require that fluid be removed irrespective of dwell time but instead based on a level of fluid in the therapy volume, pressure, vapor, humidity, moisture or other environmental indicator of the treatment volume may be used as the trigger to initiate fluid removal from the treatment volume. If the dwell time has elapsed or if the system has determined or the treatment protocol calls for fluid removal, then at step 1913, the T-valve 1935 is positioned to the wound site (see 19C), and at step 1915, the EK pump is cycled. As the EK engine pulls fluid towards the rear EK diaphragm (i.e. cycle EK pump), fluid flows from the wound site and into the pump chamber as indicated by the arrows in FIG. 19C.

At step 1917, the three way valve is positioned to the container (see FIG. 19D). At step 1919, the EK pump is cycled. As the EK engine pushes fluid towards the front EK diaphragm (i.e. cycle EK pump), fluid flows from the pump chamber in the electrokinetic pump system 1902 as shown by the arrows in FIG. 19. Fluid flows from the pump system 1902 and into the extraction chamber 1908 through the open check valve 1910b. The check valve 1910a in the drug chamber prevents the fluid from going into the drug chamber 1906.

Referring again to the flowchart 1900, the method of providing wound therapy continues by determining at step 1921 if there is more fluid to pump from the treatment volume. If so, then the steps of cycling the pump with the valve configured to draw from the wound site continues at step 1913. If not, then it is determined at step 1923 whether there is more fluid to deliver to the wound volume. If there is more fluid to deliver, then the process repeats itself starting at step 1901. If there are no more fluids to deliver to or remove from the wound therapy volume, then the method of wound therapy ends at step 1925.

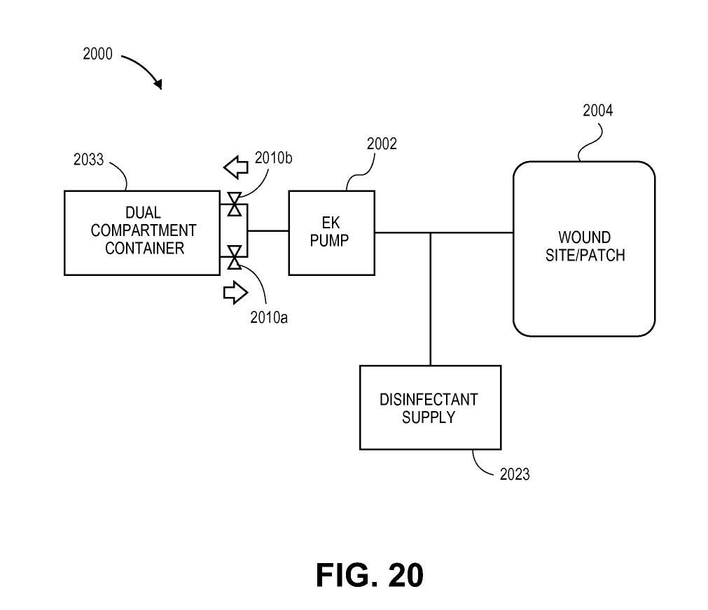

In some embodiments, a single pump system such as that shown in FIGS. 19A-19D can include a disinfectant supply and associated valves to facilitate flushing of lines after exudate is pumped from the wound site to the collection chamber. For example, as shown in FIG. 20, a system 2000 can include a wound patch 2001 connected to a single EK pump system 1902. A disinfectant supply 2023 can connect to the line between the patch and the pump. One or more valves may be provided to facilitate flushing of lines after exudate is pumped from the wound site to the collection chamber. As with system 1900, the system 2000 can include a dual compartment container 2033 and two check valves 2010a,b. The use of a disinfectant advantageously prevents the newly supplied fluid (i.e., next dose of irrigation or pharmacologically active agent according to the wound therapy regime) is not contaminated by or mixed with any remaining exudates removed from the wound site that may still be in the lines or the pump chamber.

### Negative Pressure Wound Therapy

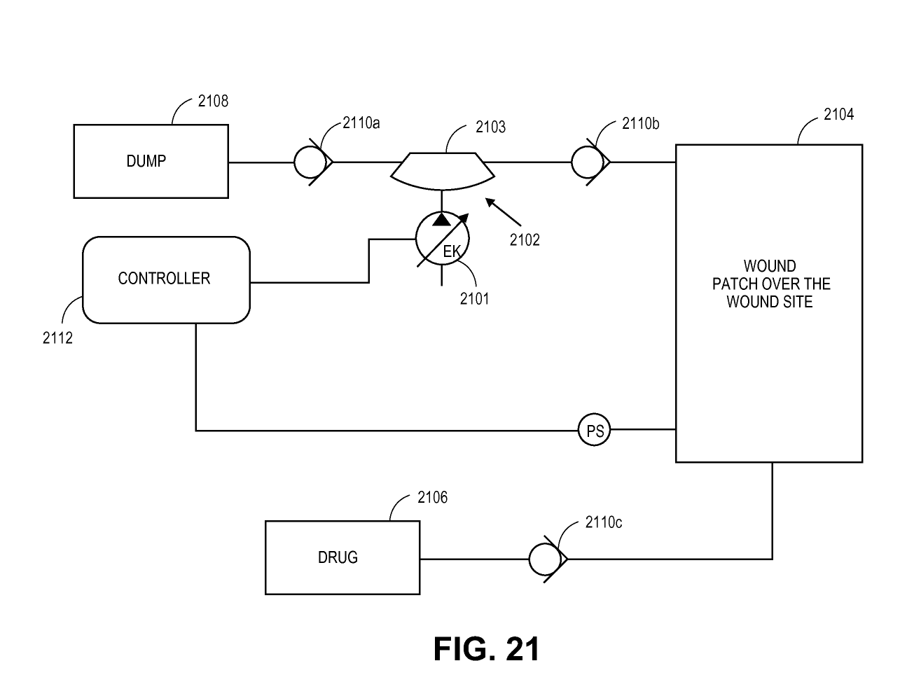

In one embodiment, shown in FIG. 21, a closed wound system 2100 can include a patch 2104 and a single electrokinetic pump system 2102 (having EK engine 2101 and pump 2103). The electrokinetic pump system 2102 can feed into an evacuation chamber 2108. The system 2100 can be configured such that the electrokinetic pump system 2102 provides enough negative pressure to pull fluid from the wound site under the patch 2104 and maintain the wound site at a predetermine negative pressure set via the cracking pressure of the inlet check valve 2110b of the pump. For example, an inlet check valve 2110b with cracking pressure of 1 psi will let the pressure inside the patch to be at negative 1 psi. Alternatively, the pressure can be set via electronically via a pressure sensor provide that it is set at a point greater than the check valve 2110b's cracking pressure.

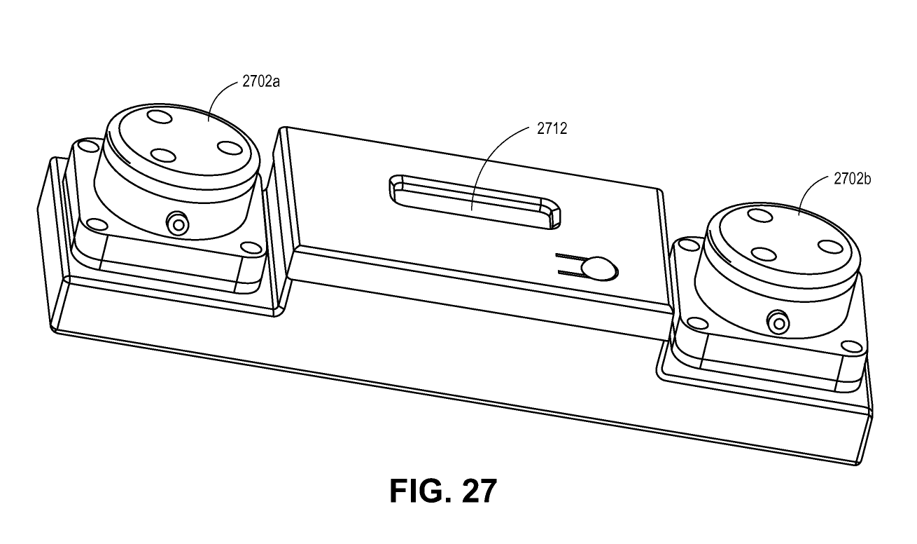

In addition, a drug supply can optionally be added to the wound patch such that the electrokinetic pump can pull the drug thru to the wound patch. Referring still to FIG. 27, there can be a first check valve 2110c between the drug reservoir and the patch and a second check valve between the patch and the pump 2110b. The first check valve can have a cracking pressure that is higher than the cracking pressure of the second check valve. For example, the first check valve can have a cracking pressure of 2 psi, while the second check valve can have a cracking pressure of 1 psi. As a result, the wound can be maintained at a negative pressure (in this case, at a pressure of −1 psi) while maintaining a continuous rinse. The constant negative pressure can advantageously improve the circulation in the wound area while the constant rinse can provide constant cleaning of the wound. A third check valve 2110c can be configured to allow treatment fluid from the reservoir 2106 to be provided to the wound area 2104 when a set pressure is reached. A controller 2112 can be configured to run a desired wound protocol.

The systems described herein can thus be used to pull a negative pressure of at least −1 psi under the patch, such as −1 psi to −5 psi. A negative pressure in this range can be strong enough to promote wound healing and allow for collapse of the patch as necessary while weak enough to avoid having the skin be pulled to the top of the patch.

Use of the electrokinetic pump assembly advantageously allows for the pulling of negative pressure even for a pump with low volume flow rates. For example, the electrokinetic pump assembly can pull negative pressures of −1 psi to −5 psi on a volume under the patch of less than 10 ml.

Such constant negative pressure over the patch can advantageously help with wound healing.

In some embodiments, negative pressure wound therapy can be combined with wet wound therapy. In such a combined system, there can be a separate evacuation pump and supply pump.

### Measuring Deflection of the Patch

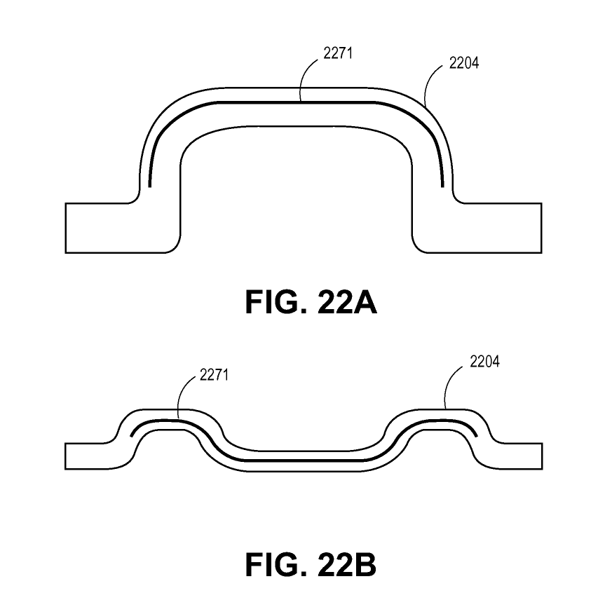

In some embodiments, a wound patch can include a sensor to detect the amount of deflection of the patch. For example, as shown in FIGS. 22A-22B, a patch 2204 can include a strain gauge 2271 or other deflection measuring device that can be embedded within or attached to one or more walls of a patch. In one aspect, the strain gauge 2271 or sensor in positioned in the location of maximum deformation of the patch during pressure changes or operations. In another aspect, shown in FIG. 22, the strain gauge 2271 extends along a length of the patch so as to more accurately measure the deformation. In another aspect, a strain gauge may be positioned in a way to span from a wall to a top surface or a wall to a base member or in other suitable locations depending upon the patch deformation properties. In another embodiment, the sensor can be a touch sensor to detect contact of a portion of the patch with tissue in the wound site.

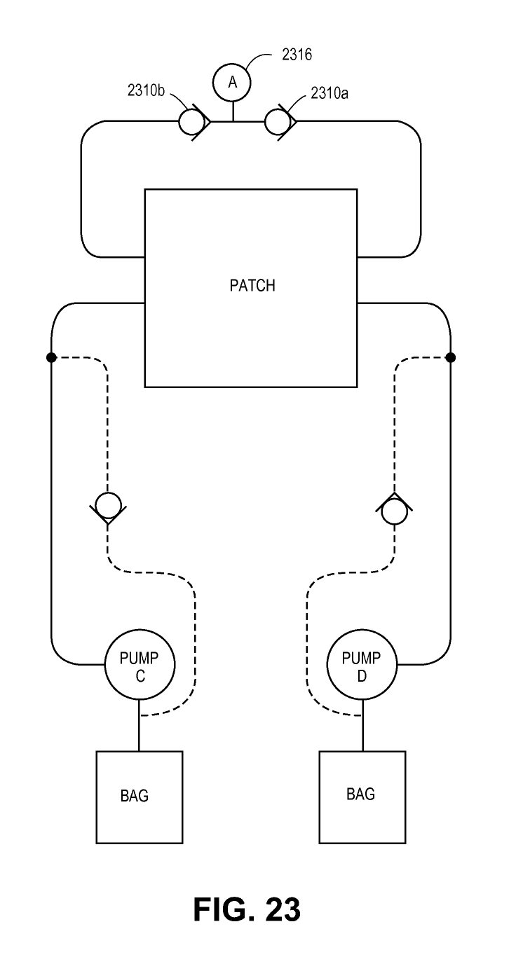

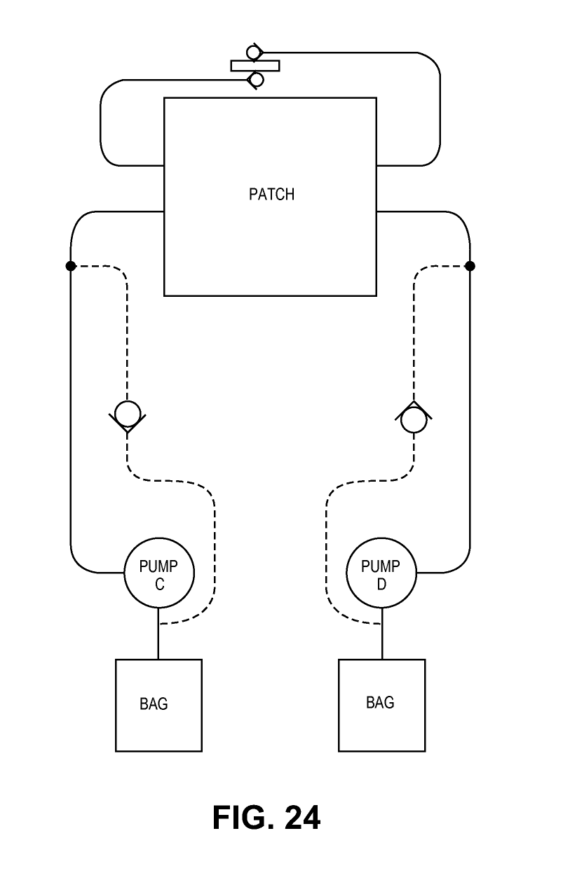

In some embodiments, referring to FIG. 23, the wound patch/pump systems can include a pressure sensor 2316 attached to the patch. A first check valve 2310a can be located on one side of the pressure sensor while a second check valve 2310b can be located on the opposite side of the pressure sensor. The system can be configured such that, if the pump is flushing fluid into the wound, the first check valve is opened. In contrast, if the system is evacuating, the other check valve is opened. In another embodiment, referring to FIG. 24, a spring-loaded switch can be used to determine the pressure.

The output from the deflection measuring device, strain gauge, or pressure sensors may be used to stop or adjust wound treatment system operations. The output may be used to cease operations if the output indicates that a portion of patch may contact the wound or that the patch integrity is breached (i.e., loss of fluid or pressure integrity in the sealed wound treatment volume). Alternatively, the output may be used to permit continued operations when the output indicates that operating conditions within the patch are remaining within normal or acceptable limits. For example, a positive or negative pressure treatment regime may continue or advance to a more aggressive level (i.e., greater pressure) if the output indicates that the wound environment is stable.

### Turning Pump on and Off

The wound pumps described herein can be configured to flush liquid through the wound or evacuate the wound based on a number of different characteristics.

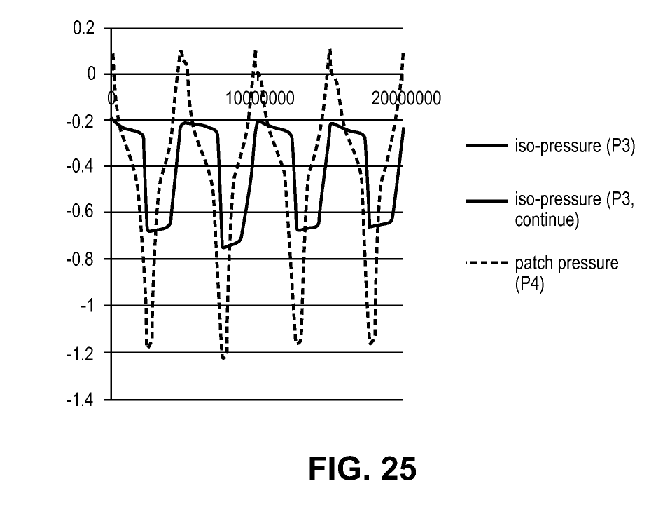

In one embodiment, the wound pump is turned on and off based entirely upon the pressure in the patch. Referring to FIG. 25, the patch is evacuated down to approximately −1 psi and then the pump is turned off. During the fluid flush, the pump is run until the pressure in the patch reaches approximately 0 psi. Advantageously, with these settings, when the patch is evacuated down to −1 psi, 70% of the volume is evacuated. Further, when fluid is pumped up to above 0 psi, 70% of the volume is delivered. In one embodiment, 7-8 ml of fluid are delivered and/or evacuated every 4-6 hours. Using the pressure in the patch to determine when to turn the pump on and off advantageously ensures that the pressure inside the patch remains low enough to create optimum circulation while not getting too low so as to cause discomfort to the patient.

The pressure inside the patch may change over time due to environmental conditions. For example, exudates from the wound could change the pressure inside the patch. In some embodiments, therefore, a pressure sensor in the patch can be configured to check the pressure continuously or at particular intervals, and the pump can be turned on or off to compensate for such pressure changes. In other embodiments, the pressure sensor can be set on an open loop to evacuate and fill the wound at set intervals in order to compensate for such changes.

In another embodiment, the patch is flushed or evacuated based upon reaching a set time limit.

In another embodiment, the patch is flushed based upon reaching a set pressure and evacuated based upon reaching a set time. In another embodiment, the patch is flushed based upon reaching a set time and evacuated based upon reaching a set pressure.

In another embodiment, the wound pump is turned on and off based upon a total volume being delivered and or evacuated based on the measured volume delivered on the drug. The total volume can be track by differential pressure sensor in the pump which measure the volume deliver each stroke. This system can advantageously compensate for pressure changes caused by the patient's movements.

### Manifold

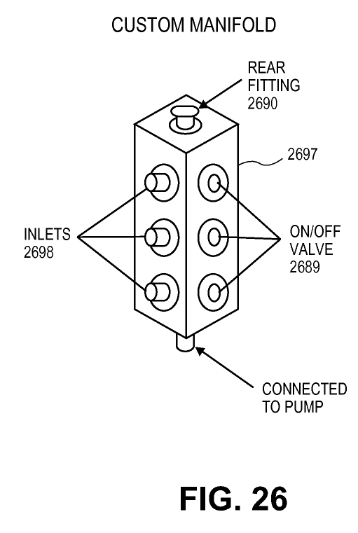

In another alternative to multiple reservoir system operations, a 3 way manifold such as that illustrated in FIG. 26 may be used. As illustrated in FIG. 26, a manifold 2697 provides three independently controlled connection inlets 2698 that may be driven by a single pump connection. On-off valves 2689 are provided to each one of the connections or inlets permitting each one to be selected individually or for all to be selected at once. A luer fitting 2690 is also provided to assist in flushing or priming operations.

### Controller and Programming

The controllers described herein may contain the instructions for operation of all pumps, valves, sensors and system components as well as the computer readable code containing the wound treatment protocol. For example, the controllers can include an electronic memory containing computer readable instructions for operating the electrokinetic pump assembly to perform a wound therapy protocol in the wound area. The wound therapy protocol can thus be pre-set and programmed into the controller.

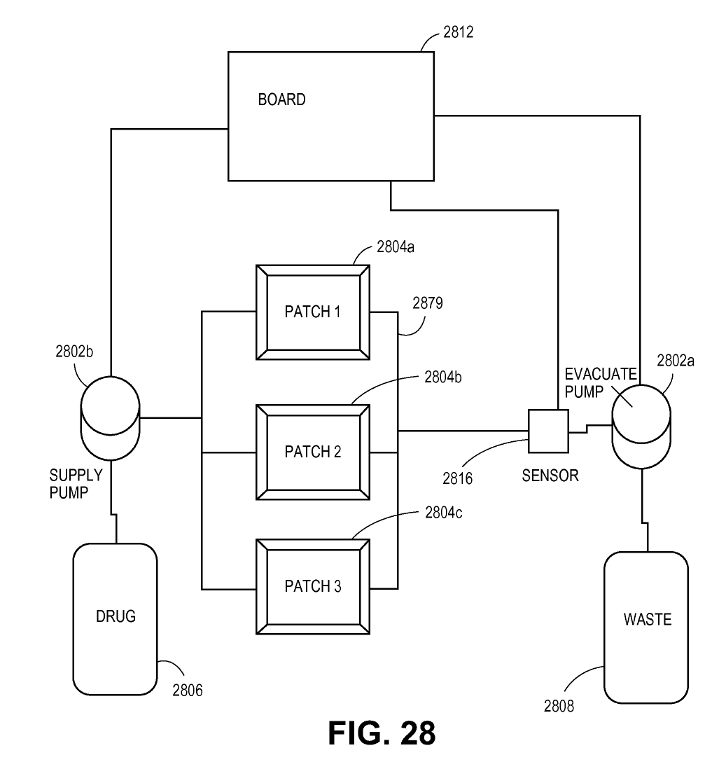

FIG. 27 illustrates a pair of electrokinetic pumps 2701 on a housing 2711 placed on either side of an electronics package including a batter power supply or AC connection and a controller. The controller is in electronic communication with the pressure sensor and the 2 pumps as shown in the system 2800 in FIG. 28. In one aspect, the controller 2812 operates the pumps in a set time value or constant duty cycle until the pressure sensor achieves a set value. Different and customized duty cycles are possible. In one exemplary operation cycle, the pumps are primed (if needed), and then the controller selects the evacuate pump. Drive signals are sent to the evacuate pump. In one embodiment, the pump will remove fluid out of the patches until the pressure sensor reaches a pressure set point. In another aspect, the pump will drive to remove fluid for a time based set point. If a pressure set point is used, the system may also use a limit for achieving the pressure set point. During a delivery cycle, the controller selects the delivery pump. The pump receives a drive signal until the pressure sensor reaches a pressure set point. As with the evacuation cycle, a time limit or other safety feature may be used to limit the operation of the delivery pump.

In some embodiments, the wound pumps described herein can be configured to be programmable by the patient or caregiver. Thus, the wound pump can be configured to deliver or remove a particular amount of fluid through bolus or basal mechanisms. For example, the pump can be configured to do a slow basal rinse exchange, such as 1 ml/hr, and then once an hour do a high bolus exchange, such as a 15-30 ml/hr high-speed rinse.

In one embodiment, the controller can be set to run the pump such that it performs a set number of strokes in a given time, such as approximately 50-100 strokes every 4 hours. In one embodiment, the pump system can be configured such that substantially the same volume of fluid is delivered to the wound site as is removed. Thus, for example, the system can include a flow sensor to monitor the amount of fluid moving in and out. In other embodiments, the controller can be configured to read pressure sensors in the system and determine whether fluid should be added or removed based upon the pressure under the patch.

In one exemplary wound protocol, fluid is pumped into the patch and allowed to sit for a period of time, such as four hours. After that period of time, the evacuation pump can remove 45-60% of the fluid, thereby always keeping fluid in the patch to keep the wound wet. More fluid can then be delivered by the delivery pump. In one embodiment, it can take approximately 15 minutes to fill the patch and 15 minutes to evacuate the patch.

The wound therapy protocol can provide for a specific amount of the treatment fluids that are to be delivered in a single dose or in multiple doses as well as the timing for such doses. The wound therapy protocol can also provide for a time duration or dwell time in which the treatments fluids are meant to remain in the wound area. The wound therapy protocol can ensure that substantially all of the fluid contents, such as waste fluid, are removed from the patch before ending the treatment or pumping in additional fluid and/or can ensure that only a particular amount, such as 40-80%, for example 45-60% of the fluid, is removed from the patch before ending the treatment or pumping in additional fluid. In one embodiment, the wound therapy protocol involves estimating the volume of fluid removed from the drug reservoir, the volume of fluid removed from the patch, and/or the volume of fluid pumped into the waste reservoir. The estimated volume of fluid delivered or removed can be based, for example, upon the number of strokes that the electrokinetic pump has performed.

The wound therapy protocol can be dependent upon the amount or type of treatment fluid being delivered from the drug reservoir to the wound site. For example, a saline solution may be used to quickly rinse the wound and therefore may be delivered and evacuated continuously over a set period of time while an antibiotic may need to be delivered and then allowed to soak for a period of time in order to be effective. The wound therapy protocol can also be dependent upon the amount of type of fluid under the patch itself. For example, if an antimicrobial solution or a growth inducing drug are used, then the contents of the fluid under the patch can be tested to determine whether enough soaking has taken place before evacuating the wound site.

The wound therapy protocol can further be set such that the pump is switched on and off after set time periods have passed. For example, the pump can be set to soak for 4 hours, evacuate, fill, and then soak for another 4 hours.

If two pump systems are used in the wound treatment system, the wound therapy protocol can be set such that the two pumps run at substantially the same time and/or on separate pumping cycles.

The wound therapy protocol can further be set so as to maintain the pressure under the patch at below 0.8 psi, such as equal to or less than 0.7 psi during all phases of the cycle. Pressures under this amount can ensure that the patch maintains a solid seal with the epidermis. Likewise, in some embodiments, the wound therapy protocol is set so as to maintain the pressure under the patch at above −5 psi, such as above −1 psi, such as above −0.5 psi. Pressures above this amount can ensure that the skin or wound area is not lifted substantially towards, into, or touching the top of the patch.

The wound therapy protocol can further be set so as to ensure that the volume of fluid pumped in the wound area at a given time is or will be less than the total volume of the inside of the patch, thereby ensuring that the patch remains in contact with the patient's skin.

In other alternatives of the wound treatment methods, the environmental conditions within a wound treatment volume may be manipulated as part of the therapy. For example, the system may include additional files, piping and/or sensors to permit an electrokinetic pump in communication with the wound treatment volume to adjust the pressure within the wound treatment volume. In such a system, a static positive pressure may be maintained within the wound treatment volume. Alternatively, a static negative pressure may be maintained within the wound treatment volume. In still further embodiments, a dynamic pressure (i.e., one with the time rate of change of pressure) may be provided in the wound treatment volume.

### Multiple Patches

In some embodiments, a wound pump system can include a single pump assembly (i.e. having a single evacuation pump system 2802a and a single fluid delivery pump system 2802b) connected to multiple patches. Referring still to FIG. 28, for example, three patches 1804a,b,c can be aligned in parallel and connected together through connection lines 2879, which then feed into the drug reservoir 2806 or the waste reservoir 2808. Using multiple patches connected together can advantageously allow for protection of a wide variety of wound sizes, shapes, and patterns.

### Disinfection or Sterilization System

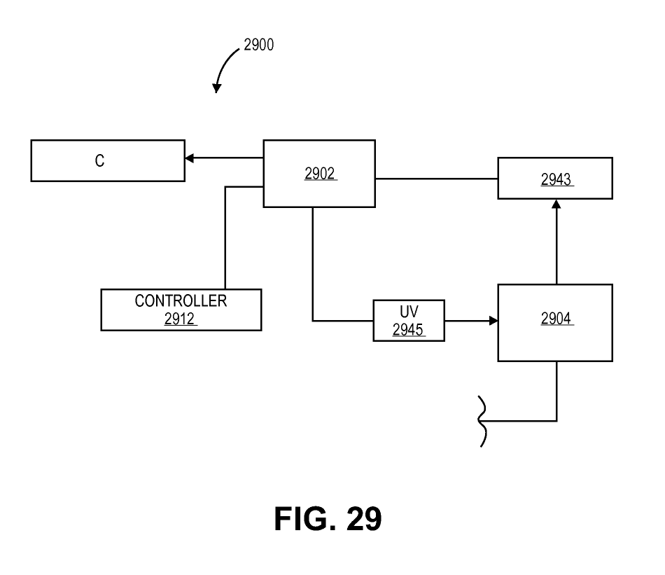

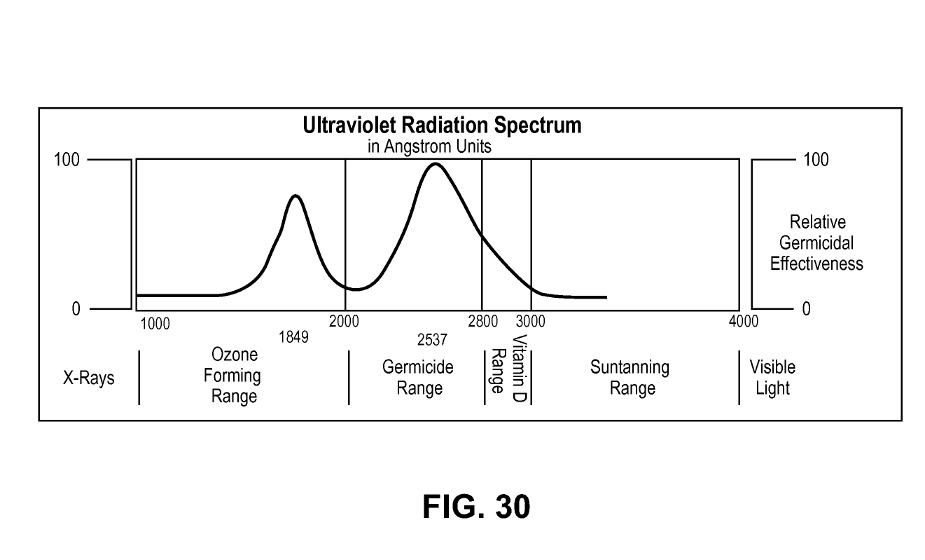

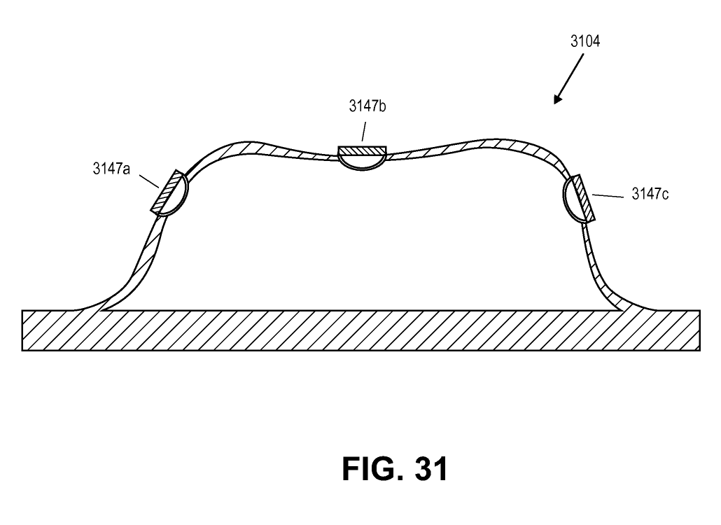

In still other aspects, the wound treatment system may include a disinfection or sterilization system or capability. FIG. 29 illustrates an alternative configuration of the wound treatment system that includes an ultraviolet treatment component (uv). The ultraviolet treatment component may be a lamp, bulb or light emitting diode. Additional details for UV light emitting diodes are provided in Enclosure D. The component is suited to the delivery of germicidal ultraviolet energy within the UV-B and UV-C band, within the range of 240 nm to 280 nm or other wavelengths suited to the particular operation required. In one embodiment the UV component provides an output at about 254 nm. FIG. 30 illustrates a curve representing the germicidal effectiveness of various wavelengths of the ultraviolet radiation spectrum. The ultraviolet treatment component may be placed to direct energy into a component such as the patch as shown in FIG. 31, which illustrates section view of a patch 3104 having three LED type bulbs 3147a,b,c positioned to emit Ultraviolet (UV) radiation into the wound treatment volume. The energy level, placement or use of shielding may be used to direct or focus the energy into the fluids of the wound treatment system and minimize the radiation exposure to the tissue in the in and around the treatment site or to the patient generally. For example, the UV component may be placed within a metal lined container or the tissue may be shielded with a suitable metalized layer, or the UV component may be with a flow tube such as those used with in-line industrial UV disinfection systems.

With reference to FIG. 29, in operation, the EK pump system 2902 of a wound treatment system 2900 draws fluid from the wound site under a patch 2904 to an inline tester 2943. The inline tester 2943 evaluates the contents of various compounds, compositions or materials in the fluids drawn from the wound site under the patch 1904. Based on the results of the tester 2943 evaluated by a user directly or by a program executed by the tester 2943, the controller 2912, or both, valves (not shown) will direct the fluid in the wound site through a circulation loop that includes the ultraviolet treatment component 2945. The treatment component may then be switched on and/or powered to the appropriate level based on the results of the tester 2943. The controller 2912 then determines how long to operate the electrokinetic pump system 2902 based upon a number of factors such as fluid flow volume, velocity past or through the UV component, the duty cycle of the UV component and the desired dosage. Depending upon the results of the tester, the system operates to provide a UV dosage to achieve a germicidal result with the fluids in the wound treatment volume. UV dosage can be, for example, between 2,500 and 30,000 μWs/cm².

### Portability and Wear

Because the pumps described herein can circulate fluid continuously or on a wound treatment protocol, the patch can be configured to be worn for more than 24 hours, such as more than 48 hours, such as 3-7 days. Advantageously, different reservoirs can be connected to the patch while it is worn to allow for different flushing liquids. For example, a reservoir containing a drug treatment for the wound could first be used, followed by a saline rinse, followed by growth-promotion drugs. Changes between types of flushing liquids can thus be made without having to change wound dressings, as is required with current technology.

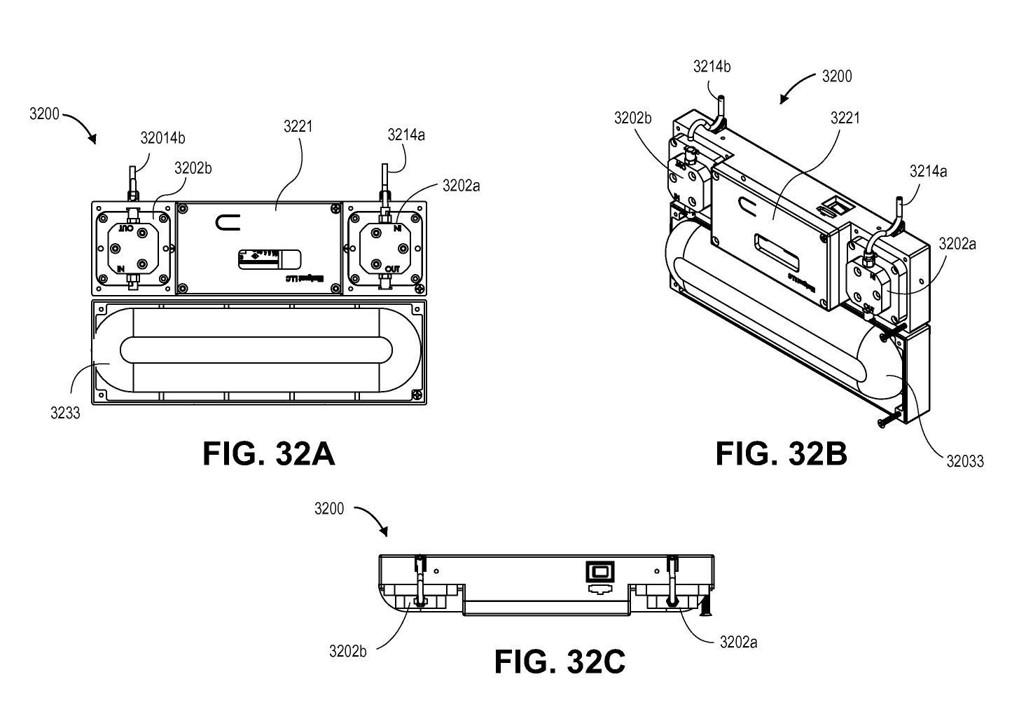

To ease portability, the wound treatment systems herein can be configured to be placed on a manifold. For example, referring to FIGS. 32A-32C, a manifold 3200 can include a container 3233, such as a split container, that can house both the supply and the waste reservoirs. The manifold 3200 can further include two pumps 3202a,b and an electronics package 3221 (including a battery and a controller). In some embodiments, the battery can be rechargeable. In other embodiments, the electronics package can include an a/c adapter. The manifold can include quick disconnects 314a,b to quickly connect and disconnect from a wound patch. The quick disconnects could alternatively or also be located between the container 3233 and the pumps 3202a,b.

Advantageously, the wound treatment systems can be lightweight, adding to the ease of portability. For example, the manifold 3200 (with the two pumps, battery, and a controller) can weigh less than 500 grams, such as less than 450 grams, such as approximately 410 grams. The pump assembly (including 2 pumps and engines) can be lightweight at less than 100 grams, such as less than 90 grams, such as approximately 75 grams.

Further, the wound treatment system can be small and compact. For example, the manifold 3200 can be less than 100 cubic inches in volume, such as less than 90, less than 80, or less than 70 cubic inches in volume. Likewise, the portion of the manifold 3200 including the pumps 3202a,b and electronics package 3221 but without the reservoirs can be less than 40 cubic inches, such as less than 30 cubic inches. In one specific embodiment, the dimensions of the manifold without the reservoirs 3233 are 8 inches in length, 2.25 inches in width, and 1.45 inches in depth.

The wound pump systems described herein are advantageously very quiet. For example, less than or equal to 50 dB, such as less than or equal to 20 dB, such as less than or equal to 10 dB, for example less than or equal to 0 dB. As such, the wound pump systems can easily be worn both while sleeping and while performing normal daily activities.

### Additional

The electrokinetic pump systems described herein can advantageously be configured to deliver a dose of fluid that is less than 1 ml, such as less than 0.5 ml, such as less than 0.1 ml. These small doses of fluid can be delivered precisely and consistently using the systems described herein. Further, incremental dose adjustments can be made over time of less than 0.5 ml, such as less than 0.1 ml. Thus, the system described herein can advantageously be used to meter fluid delivery and evacuation for wet wound therapy.

Other details of pump control, multiple reservoir configurations, and use of sensors for monitoring and controlling pump operation are further described in commonly assigned U.S. Pat. No. 7,517,440 filed Apr. 21, 2005, incorporated herein by reference.

What is claimed is:

1. A wound treatment system comprising:
a patch configured to enclose a wound area, the patch having an inlet and an outlet;
a first fluid reservoir fluidically connected to the inlet and a second fluid reservoir fluidically connected to the outlet;
an electrokinetic pump assembly, the electrokinetic pump assembly configured to pump a first treatment fluid from the first fluid reservoir into the patch through the inlet and to pump fluid from the patch through the outlet and into the second fluid reservoir; and
a controller configured for operation of the electrokinetic pump assembly, the controller having an electronic memory containing computer readable instructions for operating the electrokinetic pump assembly to perform a wound therapy protocol in the wound area.

2. The wound treatment system of claim 1, wherein the wound therapy protocol provides for an amount of the contents of the first reservoir to be delivered to the wound area.

3. The wound treatment system of claim 1, wherein the wound therapy protocol provides for a time duration that a portion of the contents of the first reservoir are to remain in the wound area.

4. The wound treatment system of claim 1, wherein the wound therapy protocol provides for a time duration for operation of the electrokinetic pump assembly to pump substantially all of the contents of the wound area to the second reservoir.

5. The wound treatment system of claim 1, wherein the wound therapy protocol provides for a time duration for the operation of the electrokinetic pump assembly depending upon the contents of the first reservoir.

6. The wound treatment system of claim 1, wherein the wound therapy protocol provides for a time duration for the operation of the electrokinetic pump assembly depending upon the fluid contents of the wound area.

7. The wound treatment system of claim 6, wherein the fluid contents of the wound area is related to the contents of the first reservoir in the wound area.

8. The wound treatment system of claim 6, wherein the fluid contents of the wound area is related to a volume of fluid in the wound area.

9. The wound treatment system of claim 1, wherein the controller is configured to estimate a volume of fluid taken from the first reservoir, a volume of fluid removed from the patch area, or a volume of fluid pumped into the second reservoir.

10. The wound treatment system of claim 9, wherein the controller is configured to estimate a volume based upon a number of cycles of the electrokinetic pump assembly operation.

11. The wound treatment system of claim 1, wherein the electrokinetic pump assembly weighs less than 75 grams.

12. The wound treatment system of claim 1, wherein the wound treatment system including the reservoirs, the pump assembly, the controller, and a power source have a volume of less than 100 cubic inches.

13. The wound treatment system of claim 1, wherein the wound treatment system is configured to be attached and carried on a patient.

14. The wound treatment system of claim 1, further comprising a container, the container including both the first and second reservoirs.

15. The wound treatment system of claim 14, wherein the container has a movable member therein to separate the first fluid reservoir from the second reservoir.

16. The wound treatment system of claim 15, wherein the movable member is configured such that the volume of the first reservoir decreases while the volume of the second reservoir increases.

17. The wound treatment system of claim 14, further comprising a second container having a third reservoir and a fourth reservoir, and wherein the second container is configured to be interchangeable with the first container such that a second treatment fluid can be pumped by the electrokinetic pump into the patch from the third reservoir and fluid can be pumped from the patch into the fourth fluid reservoir.

18. The wound treatment system of claim 1, wherein the electrokinetic pump assembly includes two electrokinetic pumps, one electrokinetic pump configured to pump fluid from the first fluid reservoir into the patch and the second electrokinetic pump configured to pump fluid from the patch into the second fluid reservoir.

19. The wound treatment system of claim 18, wherein the computer readable instructions provide for the two pumps to run at substantially the same time.

20. The wound treatment system of claim 18, wherein the computer readable instructions provide for the two pumps to run on separate pumping cycles.

21. The wound treatment system of claim 18, wherein the computer readable instructions provide for one of the two pumps to operate depending upon the contents of the first reservoir.

22. The wound treatment system of claim 18, wherein the computer readable instructions provide for one of the two pumps to operate depending upon a duration that a portion of the contents of the first reservoir has remained within the wound area.

23. The wound treatment system of claim 18, wherein the computer readable instructions provide for one of the two pumps to operate depending upon a volume of fluid contained within the wound area.

24. The wound treatment system of claim 1, further comprising a sensor configured to measure the pressure inside the patch.

25. The wound treatment system of claim 24, further comprising a controller configured to pump fluid in or out based upon the pressure.

26. The wound treatment system of claim 1, the computer readable instructions further comprising an instruction to operate the electrokinetic pump assembly such that fluid is moved in and out of the wound area at predetermined time intervals.

27. The wound treatment system of claim 1, wherein the system is configured to operate the electrokinetic pump assembly maintain the pressure under the patch at under 0.8 psi.

28. The wound treatment system of claim 1, wherein the system is configured to operate the electrokinetic pump assembly to maintain the pressure under the patch at greater than or equal to −5 psi

29. The wound treatment system of claim 1, the computer readable instructions further comprising an instruction to operate the electrokinetic pump assembly to maintain a volume of fluid in the wound area below a total volume of an enclosed wound area.

30. The wound treatment system of claim 1, the computer readable instructions further comprising an instruction to operate the electrokinetic pump assembly to maintain a volume of fluid in the wound area as defined in a wound treatment protocol.

31. The wound treatment system of claim 1, wherein the patch comprises a movable film and a protective shell.

32. The wound treatment system of claim 1, the system further comprising a bypass check valve in communication with the wound area and the second fluid reservoir with a setting to open when the pressure within the wound area reaches a set point selected to prevent loss of a sealing along the enclosed wound area.

33. The wound treatment system of claim 1, wherein the system is configured to deliver a minimum dose of the contents of the first reservoir of less than 1 ml.

34. The wound treatment of claim 33, wherein the minimum dose has a volume of less than 0.5 ml.

35. The wound treatment system of claim 34, wherein the minimum dose has a volume of less than 0.1 ml.

36. The wound treatment system of claim 1, wherein the system is configured to deliver a dose of the contents of the first reservoir with an incremental dose adjustment of less 0.5 ml.

37. The wound treatment system of claim 1, wherein the system is configured to deliver a dose of the contents of the first reservoir with an incremental dose adjustment of less 0.1 ml.

38. The wound treatment system of claim 1, further comprising a battery configured to run the electrokinetic pump assembly.

39. The wound treatment system of claim 38, wherein the battery is configured to run the electrokinetic pump assembly for over 48 hours without charging.

40. The wound treatment system of claim 38, wherein the battery, patch, and pump assembly weigh less than 450 grams.

41. The wound treatment system of claim 38, wherein the battery is a rechargeable battery.

42. The wound treatment system of claim 1, further comprising an AC adapter for powering the electrokinetic pump assembly.

43. The wound treatment system of claim 1, further comprising at least one quick disconnect mechanism configured to disconnect the patch from the first and second fluid reservoirs such that third and fourth fluid reservoirs can be attached to the patch.

44. The wound treatment system of claim 43, wherein the quick disconnect is between the patch and the electrokinetic pump assembly.

45. The wound treatment system of claim 43, wherein the quick disconnect is between the electrokinetic pump assembly and the first and second reservoirs.
